SEQUENCE LISTING (19 sequences)

SEQ ID NO: 1 - protein, 679 aa, Homo Sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 679 aa, Homo Sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKADNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVADCSGNFCLFRSETKDLLFRDDTVCLAKLHDRNTYEKYLGEEYVKAVGNLRKCSTSSLLEACTFRRP

SEQ ID NO: 3 - DNA, 2040 bp, Homo sapiens
gtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataaggctgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtagctgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 4 - DNA, 42 bp, Artificial Sequence
  oligonucleotide
ggcagaaaactacaataaggctgataattgtgaggatacacc

SEQ ID NO: 5 - DNA, 42 bp, Artificial Sequence
  Oligonucleotide CF 157
ggtgtatcctcacaattatcagccttattgtagttttctgcc

SEQ ID NO: 6 - DNA, 45 bp, Artificial Sequence
  Oligonucleotide CF 158
gcacctatttggaagcaacgtagctgactgctcgggcaacttttg

SEQ ID NO: 7 - DNA, 45 bp, Artificial Sequence
  Oligonucleotide CF 159
caaaagttgcccgagcagtcagctacgttgcttccaaataggtgc

SEQ ID NO: 8 - DNA, 24 bp, Artificial Sequence
  Oligonucleotide DS 181
ctcaaccaggcccaggaacatttt

SEQ ID NO: 9 - DNA, 19 bp, Artificial Sequence
  Oligonucleotide DS182
agagaccaccgaagactgc

SEQ ID NO: 10 - DNA, 19 bp, Artificial Sequence
  Oligonucleotide DS 183
aaccactgcagatttgatg

SEQ ID NO: 11 - DNA, 18 bp, Artificial Sequence
  Oligonucleotide DS 184
gccagagccccgaatcac

SEQ ID NO: 12 - DNA, 22 bp, Artificial Sequence
  Oligonucleotide DS 185
atttttcatatgtgtttctgtc

SEQ ID NO: 13 - DNA, 20 bp, Artificial Sequence
  oligonucleotide DS 186
ttcacaaaggccacatctcc

SEQ ID NO: 14 - DNA, 18 bp, Artificial Sequence
  Oligonucleotide DS 187
caaaataccctgcctctg

SEQ ID NO: 15 - DNA, 17 bp, Artificial Sequence
  oligonucleotide M13-F
gtaaaacgacggccagt

SEQ ID NO: 16 - DNA, 16 bp, Artificial Sequence
  oligonucleotide M13.R
aacagctatgaccatg

SEQ ID NO: 17 - protein, 679 aa, Artificial Sequence
  Transferrin mutant
VPDKTVRWCAVSEHEATKCQSFRDHMKSVIPADGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKADNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVADCSGNFCLFRSETKDLLFRDDTVCLAKLHDRNTYEKYLGEEYVKAVGNLRKCSTSSLLEACTFRRP

SEQ ID NO: 18 - DNA, 2359 bp, Artificial Sequence
  DNA construct
cttaagagtccaattagcttcatcgccaataaaaaaacaagcttaacctaattctaacaagcaaagatgttgttgcaagcttttttgtttttgttggctggttttgctgctaaaatttctgctgttccagataaaacagttagatggtgtgctgtttctgaacatgaagctactaaatgtcaatcttttagagatcatatgaaatctgttattccatctgatggtccatctgttgcttgtgttaaaaaagcttcttatttggattgtattagagctattgctgctaatgaagctgatgctgttactttggatgctggtttagtttatgatgcttatttggctccaaacaatttgaaaccagttgttgctgaattttatggttctaaggaagatccacaaactttttattatgctgtagccgttgtaaaaaaggattcaggttttcaaatgaatcaattgagaggtaaaaaatcttgtcatactggtttaggtagatctgctggatggaatattccaattggtttgttgtattgtgatttgccagaaccaagaaaaccattggaaaaagctgttgctaattttttttctggttcttgtgctccatgtgctgatggtacagattttccacaattgtgtcaattatgtccaggttgtggttgttctactttgaatcaatattttggttattctggtgcttttaaatgtttgaaagatggtgctggtgatgttgcttttgttaaacattctactatttttgaaaatttggcaaacaaagctgatagagatcaatatgaattgttgtgtttggataatactagaaaaccagttgatgaatataaagattgtcatttggctcaagttccatctcatactgttgttgctagatctatgggtggtaaagaagatttgatttgggaattgttgaatcaagctcaagaacattttggtaaagataaatctaaagaatttcaattgttttcttctccacatggtaaagatttgttgtttaaagattctgctcatggttttttgaaagttccaccaagaatggatgctaaaatgtatttgggttatgaatacgttactgctattagaaatttgagagaaggtacttgtccagaagctccaactgatgaatgtaaaccagttaaatggtgtgctttgtctcatcatgaaagattaaaatgtgatgaatggtctgttaattctgttggtaaaattgaatgtgtttctgctgaaactacagaagattgtattgctaaaattatgaatggtgaagctgatgctatgtctttagatggtggttttgtatatattgctggtaaatgtggtttagttccagttttggctgaaaattataacaaagctgataattgtgaagatactccagaagctggttattttgctgttgctgttgttaaaaaatctgcttctgatttgacttgggataatctaaaaggaaaaaagagttgccatacagctgttggaagaacagccggatggaacattccaatgggattgctatacaacaaaattaatcattgtagatttgatgaatttttttctgaaggttgtgctccaggttctaaaaaagattcttctttgtgtaaattgtgtatgggttctggattgaatttgtgtgaaccaaacaacaaggaaggttattatggttatactggtgcttttagatgtttagttgaaaaaggtgatgttgcttttgttaaacatcaaacagttccacaaaatactggtggtaaaaatccagatccatgggctaaaaatttgaatgaaaaagattacgaattactatgtttagatggtacaagaaagccagttgaggaatacgctaattgtcatttagctagagcaccaaatcatgctgttgttactagaaaagataaagaagcttgtgttcataaaattttgagacaacaacaacatttgtttggttctaatgttgctgattgttctggtaatttttgtttgtttagatctgaaactaaagatttattgtttagagatgatactgtttgtttggctaaattgcatgatagaaatacttatgaaaaatatttgggtgaagaatacgttaaagctgttggtaatttgagaaaatgttctacttcttctttgttggaagcttgtacttttagaaggccataataagcttaattcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgc

SEQ ID NO: 19 - protein, 19 aa, Artificial Sequence
  Invertase leader
MLLQAFLFLLAGFAAKISA

(19) United States
(12) Patent Application Publication
(10) Pub. No.: US 2010/0222267 A1
(43) Pub. Date: Sep. 2, 2010

(54) RECOMBINANT TRANSFERRIN MUTANTS

(75) Inventors: Christopher John Arthur Finnis, Nottingham (GB); Darrell Sleep, Nottingham (GB); Joanna Hay, Leicestershire (GB)

Correspondence Address: NOVOZYMES NORTH AMERICA, INC., 500 FIFTH AVENUE, SUITE 1600, NEW YORK, NY, 10110, US

(73) Assignee: Novozymes Biopharma UK Limited., Bagsvaerd (DK)

(21) Appl. No.: 12/664,454

(22) PCT Filed: Jun. 13, 2008
(86) PCT No.: PCT/EP2008/057508

§ 371 Date: Dec. 14, 2009

Related U.S. Application Data
(60) Provisional application No. 60/944,554, filed on Jun. 18, 2007.

(30) Foreign Application Priority Data
Jun. 13, 2007 (EP) 0711424.2

(51) Int. Cl. A61K 38/40; C07K 14/79; C07H 21/04; C12N 15/63; C12P 21/06; C12N 5/00; C12N 5/02

(52) U.S. Cl. 514/12; 530/394; 536/23.5; 435/320.1; 435/455; 435/69.1; 435/325; 435/389

(57) ABSTRACT

The present invention provides a recombinant protein comprising the sequence of a transferrin mutant, wherein Ser415 is mutated to an amino acid which does not allow glycosylation at Asn413 and/or wherein Thr613 is mutated to an amino acid which does not allow glycosylation as Asn611. It also provides polynucleotides encoding the same and methods of making and using said recombinant protein.

### FIELD OF THE INVENTION

The present application relates to recombinant transferrin mutants and proteins comprising the sequence of these, particularly mutants that avoid N-linked glycosylation and retain the biological activity of the wild-type protein. The present application also relates to polynucleotides encoding a recombinant protein comprising the sequence of a transferrin mutant and methods of making and using the recombinant protein.

### BACKGROUND OF THE INVENTION

The listing or discussion of a prior-published document in this specification should not necessarily be taken as an acknowledgement that the document is part of the state of the art or is common general knowledge.

It is estimated that there are more than 370 new biotechnology medicines in the pipeline. Producing biotech drugs is a complicated and time-consuming process. Cells must be grown in large stainless-steel fermentation vats under strictly maintained and regulated conditions. In some cases the proteins are secreted by the cells; in other cases the cells must be broken open so the protein can be extracted and purified. Once the method is tested, devised and scaled up, the biotech medicines can be produced in large batches. This is done by growing host cells that have been transformed to contain the gene or antibody of interest in carefully controlled conditions in large stainless-steel tanks. The cells are kept alive and stimulated to produce the target proteins through precise culture conditions that include a balance of temperature (which can often vary by no more than one degree Celsius), oxygen, acidity (if pH levels change by even a small fraction, cells can easily die), media components and other variables. After careful culture in the appropriate media or serum (the duration varies depending on the protein produced and the nature of the organism), the proteins are isolated from the cultures, stringently tested at every step of purification, and formulated into pharmaceutically-active products. All of these procedures are in strict compliance with Food and Drug Administration (FDA) regulations. (http://www.bio.org/pmp/factsheet1.asp, “A Brief Primer on Manufacturing Therapeutic Proteins”).

There are many varied types of cell culture media that can be used to support cell viability, for example DMEM medium (H. J. Morton, 1970, In Vitro, 6, 89), F12 medium (R. G. Ham, 1965, Proc. Natl. Acad. Sci. USA, 53, 288) and RPMI 1640 medium (J. W. Goding, 1980, J. Immunol. Methods, 39, 285; JAMA, 1957, 199, 519). Such media (often called “basal media”), however, are usually seriously deficient in the nutritional content required by most animal cells. Typically, serum must be added to the basal media to overcome these deficiencies. Generally, foetal bovine serum (FBS, harvested from the foetuses of cows), human serum, porcine serum and horse serum are used in significant concentrations.

While the use of serum is desirable, and often necessary, for proper cell growth, it has several disadvantages. It is difficult to obtain serum with consistent growth characteristics. Further, the biochemical complexity of the serum can complicate the downstream processing of the proteins of interest, therefore raising the production costs. In an attempt to solve this problem the serum has been removed and specific components have been added instead.

One of these components is transferrin. Human serum transferrin (HST) is the major iron-binding protein in normal human plasma, and is present at about 2-4 g/l (van Campenhout et al, 2003, Free Radic. Res., 37, 1069-1077). Physiologically, it functions in the safe transport of iron from sites of absorption and storage to sites of utilisation, such as developing red blood cells. Its high affinity for iron reduces the risk of damaging effects from iron-catalysed free-radical reactions (von Bonsdorff et al, 2001, Biologicals, 29, 27-37) in the extracellular environment, and the consequent low free-iron concentration is bacteriostatic to many organisms (von Bonsdorff et al, 2003, FEMS Immunol. Med. Microbiol., 37, 45-51); it may also have more direct antibacterial effects (Ardehali et al, 2003, J. Biomed. Mater. Res. A, 66, 21-28).

HST is a monomeric glycoprotein of molecular weight about 80 kDa with the capacity to bind two ferric ions very tightly, but reversibly. It comprises two globular lobes (referred to as the N-lobe and C-lobe) each made up of two sub-domains separated by a deep cleft, which contains the binding site for a ferric ion and a synergistic carbonate anion. In the vast majority of cell types, iron is acquired by binding of iron-laden holo-transferrin to a specific transferrin receptor (TfR), followed by endocytosis of the Fe³⁺/HST/TfR complex. Iron is released in the acidic conditions of the endosome, after which the HST/TfR complex is returned to the cell surface, from where the iron-free apo-transferrin is released back to the circulation (MacGillivray et al, 1998, Biochemistry, 37, 7919-7928; Hirose, 2000, Biosci. Biotechnol. Biochem., 64, 1328-1336; Hemadi et al, 2004, Biochemistry, 43, 1736-1745).

HST is produced in the liver as a 698-residue protein. A 19-residue leader sequence is removed during secretion to produce a mature glycoprotein of approximately 80 KDa, having the amino acid sequence of SEQ ID No. 1. The approximately 75 KDa polypeptide chain of mature transferrin contains 19 disulphide bonds and has a predicted pl of 6.64. The N-lobe and C-lobe are formed from residues 1-331 and 338-679, respectively (Steinlein et al, 1995, Protein. Expr. Purif., 6, 619-624). The C-lobe contains the two N-linked glycosylation sites at Asn413 and Asn611 (underlined in SEQ ID No. 1 as shown above). An O-linked glycosylation site at serine 32 has also been identified in N-lobe transferrin produced by recombinant expression from baby hamster kidney cells (Gomme et al, 2005, Drug Discov. Today, 10, 267-273) and the yeast, P. pastoris (Bewley et al, 1999, Biochemistry, 38, 2535-2541).

Any animal or mammalian transferrin may be used in cell culture media, such as HST or bovine serum transferrin (BST).

However, there is an increased focus on the risk of possible contamination of cell cultures with pathogens when using animal-derived components, such as transferrin. In the case of BST, the presence of prions thought to be responsible for mad-cow disease (BSE) is in particular a problem associated with the production of BST by blood fractionation. For HST there is a risk of possible contamination by hepatitis and immunodeficiency viruses like HIV, when HST is produced by blood fractionation.

A recombinant transferrin medium is an excellent alternative to standard serum-containing media for the cultivation of cells. It has several advantages, which include better definition of the composition, and very importantly no risk for contamination with pathogens originating from animals.

Due to the increased focus on blood transferred diseases from humans and animals, there has been an increased focus on finding a medium free from animal-derived transferrin, and other traditionally animal-derived components, having cultivation ability comparable to that of the conventional serum-containing medium. There is a continuing need in the art for cell culture media that include no risk of using with regard to transferral of diseases but provide all of the necessary nutrients and growth factors, at suitable concentrations, to optimize the growth of the cells.

Most of the effort in recent years has been to develop serum-free media by supplementing the basal media with appropriate nutrients to avoid the addition of serum, without sacrificing cell viability and/or cell growth and/or protein production. Examples of such components include bovine transferrin and human transferrin; bovine albumin and human albumin; certain growth factors derived from natural (animal) or recombinant sources, including epidermal growth factor (EGF) or fibroblast growth factor (FGF); lipids such as fatty acids, sterols and phospholipids; lipid derivatives and complexes such as phosphoethanolamine, ethanolamine and lipoproteins; protein and steroid hormones such as insulin, insulin like growth factor (IGF), hydrocortisone and progesterone; nucleotide precursors; and certain trace elements (reviewed by Waymouth, C., in: Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc., pp. 23-68 (1984), and by Gospodarowicz, D., Id., at pp 69-86 (1984)).

However, most of the prior art listed herein are still describing media comprising animal derived components.

Bowman & Yang (U.S. Pat. No. 5,026,651, granted in 1991) disclose the isolation of a cDNA sequence that encodes HST. The sequence disclosed therein is incorporated into this application by reference. Thus, it has been technically possible for some time to construct HST-coding vectors and express them to produce recombinant HST. However, as discussed above in respect of SEQ ID No. 1, the sequence of HST includes two consensus sites for N-linked glycosylation. Lau et al (1983, J. Biol. Chem., 258, 15225-15260) reported that oligosaccharyl transferase catalyzes the transfer of saccharide chains to an asparagine residue contained within the sequence -Asn-X-Thr/Ser- of proteins, where X is any amino acid. The sequence of HST contains two such consensus sequences, starting with the amino acids N413 and N611, respectively, both of which are recognised by oligosaccharyl transferase resulting in N-linked glycosylation at N413 and N611. The nature of the recombinant host cell chosen has a marked effect on the level and type of glycosylation of the HST product and this can lead to the production of a heterogeneous HST product with potentially undesirable antigenic effects in humans. In other words, HST produced recombinantly in non-human cells can be significantly differently glycosylated compared to serum-derived HST.

### SUMMARY OF THE INVENTION

In a first aspect of the present invention, there is provided a recombinant protein comprising the sequence of a transferrin mutant, wherein Ser415 is mutated to an amino acid which does not allow glycosylation at Asn413. Ser415 may be mutated to an amino acid that does not substantially reduce the biological function of the transferrin mutant. For example, Ser415 may be mutated to an amino acid that is a conserved amino acid, such as glycine or alanine. Alanine may be preferred.

A recombinant protein comprising the sequence of a transferrin mutant according to the first aspect of the invention may also comprise a mutation to Asn611 such that it is also mutated to an amino acid which does not allow glycosylation at that location. Asn611 may be mutated to an amino acid that does not substantially reduce the biological function of the transferrin mutant. For example, Asn611 may be mutated to a conserved amino acid, or it may be mutated to aspartic acid or glutamine.

A recombinant protein comprising the sequence of a transferrin mutant according to the first aspect of the invention may also comprise a mutation to Val612 such that it is mutated to an amino acid which does not allow glycosylation at Asn611. Val612 may be mutated to an amino acid that does not substantially reduce the biological function of the transferrin mutant. For example, Val612 may be mutated to proline, cysteine or tryptophan.

In a second aspect of the present invention, there is provided a recombinant protein comprising the sequence of a transferrin mutant, wherein Thr613 is mutated to an amino acid which does not allow glycosylation at Asn611. Thr613 may be mutated to an amino acid that does not substantially reduce biological function of the mutant. Thr613 may be mutated to a conserved amino acid, such as glycine, valine, alanine or methionine. Alanine may be preferred.

A recombinant protein comprising the sequence of a transferrin mutant according to the sec- and aspect of the invention may also comprise a mutation to Asn413 such that it is also mutated to an amino acid which does not allow glycosylation at that location. Asn413 may be mutated to an amino acid that does not substantially reduce the biological function of the mutant. For example, Asn413 may be mutated to a conserved amino acid, or it may be mutated to aspartic acid or glutamine.

A recombinant protein comprising the sequence of a transferrin mutant according to the second aspect of the invention may also comprise a mutation to Lys414 such that it is also mutated to an amino acid which does not allow glycosylation at Asn413. Lys414 may be mutated to an amino acid that does not substantially reduce the biological function of the mutant. For example, Lys414 may be mutated to proline, cysteine or tryptophan.

In a third aspect of the present invention, there is provided a recombinant protein comprising the sequence of a transferrin mutant wherein Ser415 is mutated in accordance with the first aspect of the present invention and wherein Thr613 is mutated in accordance with the second aspect of the present invention. A recombinant protein comprising the sequence of a transferrin mutant according to the third aspect of the invention may also comprise mutations at any, or all, of Asn413, Lys414, Asn611 and/or Val612, in the manner defined above for the first and second aspects of the present invention.

A preferred embodiment of the third aspect of the present invention may be a protein comprising, or consisting of, the sequence of a human transferrin protein that has the mutations S415A, T613A. An exemplary sequence for this protein is given as SEQ ID No. 2.

S415A and T613A mutations within the two -N-X-S/T- recognition sequences for N-linked glycosylation.

In a fourth aspect of the present invention, there is provided a recombinant protein comprising the sequence of a transferrin mutant, where in addition to a mutation in Ser415 to an amino acid that does not allow glycosylation at Asn413 and/or a mutation in Thr613 to an amino acid that does not allow glycosylation in Asn 611, at least one further mutation is introduced that reduced O-linked glycosylation of the protein. A preferred example of at least one mutation that reduces O-linked glycosylation is a mutation at Ser32, such as S32A or S32C.

In a fifth aspect of the present invention, there is provided a polynucleotide comprising a sequence that encodes a protein comprising the sequence of a transferrin mutant as defined above by any one of the first, second, third or fourth aspects of the present invention. For example, a polynucleotide according to the fifth aspect of the present invention may encode a protein comprising, or consisting of, the sequence of SEQ ID No. 2. Such a polynucleotide to sequence may have the sequence of SEQ ID No. 3.

In SEQ ID No. 3, the S415 and T613 codons of a human transferrin cDNA (derived from National Centre for Biotechnology Information nucleotide sequence NM 001063) are altered to the alanine codon GCT, which is preferred in S. cerevisiae (37%, http://www.yeastgenome.org/codon_usage.shtml). To achieve this, the AGC codon of serine 415 was altered to GCT at positions 1243 to 1245, and the ACT codon for threonine 613 was altered to GCT by mutating the adenine to a guanine at position 1837.

A polynucleotide according to the fifth aspect of the invention may comprise a secretion leader sequence. Thus, the sequence that encodes a recombinant protein comprising the sequence of a transferrin mutant may be operably linked to a polynucleotide sequence that encodes a secretion leader sequence. For example, the sequence that encodes a recombinant protein comprising the sequence of a transferrin mutant may be operably linked, at its 5′ end, to the 3′ end of a polynucleotide sequence that encodes a secretion leader sequence.

In a sixth aspect of the present invention, there is provided a plasmid comprising a polynucleotide according to the fifth aspect of the invention. In one embodiment, the plasmid may further comprises a polynucleotide sequence that encodes protein disulphide isomerase. The plasmid may be a 2 μm plasmid.

In a seventh aspect of the present invention, there is provided a use of a polynucleotide or plasmid according to the fifth or sixth aspects of the present invention to transform a host cell and thereby produce a recombinant protein comprising the sequence of a transferrin mutant according to any one of the first, second, third or fourth aspects of the invention.

In an eighth aspect of the present invention, there is provided a method of producing a host cell capable of expressing a recombinant protein comprising the sequence of a transferrin mutant according to any one of the first, second, third or fourth aspects of the invention, the method comprising providing a polynucleotide or plasmid according to the fifth or sixth aspects of the present invention; providing a host cell; transforming the host cell with the polynucleotide or plasmid; and selecting for a transformed host cell.

In a ninth aspect of the present invention, there is provided a method of producing a recombinant protein comprising the sequence of a transferrin mutant according to any one of the first, second, third or fourth aspects of the invention, the method comprising providing a host cell containing a polynucleotide or plasmid according to the fifth or sixth aspects of the present invention; and culturing the host cell under conditions that allow for the expression of the recombinant protein comprising the sequence of a transferrin mutant. The method may furthen comprise the step of isolating the expressed recombinant protein. The method may also further comprise the step of formulating the isolated recombinant protein with a carrier or diluent and optionally presenting the formulated protein in a unit dosage form, or the step of lyophilising the isolated recombinant protein.

The host cell defined by the seventh, eighth or ninth aspects of the invention may be any is type of host cell. It may, for example, be a bacterial or yeast (or other fungal) host cell. Bacterial host cells may be particularly useful for cloning purposes. Yeast host cells may be particularly useful for expression of genes present in the plasmid. In one embodiment the host cell is a yeast cell, such as a member of the Saccharomyces, Kluyveromyces, or Pichia genus, such Saccharomyces cerevisiae, Kluyveromyces lactis, Pichia pastoris and Pichia membranaefaciens, or Zygosaccharomyces rouxii, Zygosaccharomyces bailii, Zygosaccharomyces fermentati, or Kluyveromyces drosphilarum. In one further embodiment the host cell may be a fungal cell, such as Aspergillus niger, Aspergillus oryzae, Trichoderma, Fusarium venenatum, Pichia angusta or Hansenula polymorpha.

In a tenth aspect of the present invention, there is provided a mammalian cell culture medium comprising a recombinant protein comprising the sequence of a transferrin mutant according to any one of the first, second, third or fourth aspects of the invention and one or more components selected from the group consisting of; glutamine, insulin, insulin-like growth factors, albumin, ethanolamine, fetuin, vitamins, lipoprotein, fatty acids, amino acids, sodium selenite, peptone and antioxidants.

In an eleventh aspect of the present invention, there is provided a method of culturing mammalian cells, said method comprising incubating the cells in a cell culture media comprising a recombinant protein comprising the sequence of a transferrin mutant according to any one of the first, second, third or fourth aspects of the invention and one or more components selected from the group consisting of; glutamine, insulin, insulin-like growth factors, albumin, ethanolamine, fetuin, vitamins, lipoprotein, fatty acids, amino acids, sodium selenite, peptone and antioxidants.

In a twelfth aspect of the present invention, there is provided a pharmaceutical composition comprising a recombinant protein comprising the sequence of a transferrin mutant according to any one of the first, second or third aspects of the invention and a pharmaceutically acceptable carrier.

### DETAILED DESCRIPTION OF THE INVENTION

The present invention relates to recombinant protein comprising the sequence of a transferrin mutant. By “recombinant” we mean a protein that has been produced by expression of a genetically-modified (i.e. non-natural) gene sequence in a host cell. In general, a recombinant protein of this invention is produced by transforming a suitable host cell with a nucleic acid construct encoding the transferrin mutant, culturing the transformed host cell under conditions appropriate for expression and recovering the recombinant protein comprising the sequence of a transferrin mutant expressed by the cell.

Mutant forms of transferrin can be produced by standard techniques of site-directed mutagenesis and the like, such as reported in the examples below.

Recombinant proteins comprising the sequence of a transferrin mutant of the invention are defined by reference to the mutation and/or prevention of glycosylation of particular amino acids in the HST sequence defined by SEQ ID No. 1 (inter alia, Ser415 of SEQ ID No. 1; Asn611 of SEQ ID No. 1; Val612 of SEQ ID No. 1; Thr613 of SEQ ID No. 1; Asn413 of SEQ ID No. 1; Lys414 of SEQ ID. No. 1). However, the present invention is based on an improved understanding of the function and role of serine and threonine amino acids in the two glycosylation site consensus sequences of transferrin, and it is not limited in its application to introducing the specified mutations into the entire and exact sequence of a transferrin protein defined by SEQ ID No. 1.

HST has many variants, as revealed by isoelectric focusing (IEF) (Constans et al, 1980, Hum. Genet., 55, 111-114; Namekata et al., 1997, Hum. Genet., 100, 457-458). At least 22 functional variants have been detected by electrophoresis following neuraminidase treatment and saturation with iron. These variants differ in their primary amino acid sequence (the first determinant), which can be genetically characterised to define specific amino acid substitutions or deletions. Further variation occurs with differences in iron content (second determinant) and differences in the N-linked glycan chain (third determinant (de Jong et al., 1990, Clin. Chim. Acta, 190, 1-46)).

In European populations more than 95% of the population have been designated as having the TfC phenotype (de Jong et al, 1990, op. cit.). In 1987 the total number of C-variants was claimed to be 16. The two major variants overall have been tentatively designated TfC₁ and TfC₂, of which TfC₁ has been calculated to be most common, occurring at a frequency of approximately 0.74 to 0.82 (Kuhnl & Spielmann, 1978, Hum. Genet., 43, 91-95; Kuhnl & Spielmann, 1979, Hum. Genet., 50, 193-198; Weidinger et al, 1980, Z. Rechtsmed., 85, 255-261). A C/T base substitution at codon 570 replaced proline in TfC₁ with serine in TfC₂. From eskimos to aboriginals, the C₁ subspecies has been identified as the outstanding prominent transferrin, which suggests a strong selectional advantage (de Jong et al, 1990, op. cit.). The TfC₁ phenotype is heterogeneous and can be divided into two sub-types based on analysis of restriction fragment length polymorphisms (RFLP) (Beckman et al, 1998, Hum. Genet., 102, 141-144).

SEQ ID No. 1 is based on the mature TfC₁ protein sequence, and (in SEQ ID No. 2) we have presented the modified sequence in which serine 415 and threonine 613 within the oligosaccharyl transferase recognition sequences were altered to alanine residues to prevent N-linked glycosylation at the Asn413 and Asn611 sites, respectively.

In view of the variability of transferrin, even within the human population, and furthermore since the present invention is based on an improved understanding of the function and role of serine and threonine amino acids in the two glycosylation site consensus sequences of transferrin, which is not limited in its application to the entire and exact sequence of a transferrin protein defined by SEQ ID No. 1, then the skilled reader will appreciate that the term “transferrin” as used herein may be used to refer to other transferrin proteins in addition to the protein defined by SEQ ID No. 1. For example, other natural and non-natural transferrin sequences may also be encompassed by the term “transferrin”, in which they contain equivalent amino acids to Ser415 and/or Thr613 of SEQ ID No. 1.

An equivalent amino acid to Ser415 and/or Thr613 of SEQ ID No. 1 is a serine or threonine residue that is present in a N-linked glycosylation consensus site (i.e. within a sequence that is recognised by an oligosaccharyl transferase enzyme) of a transferrin protein, typically having the sequence (in the N- to C-direction) of N-X-S or N-X-T wherein X is any amino acid, such as lysine or valine, and typically not cysteine, tryptophan or proline. However, the equivalent amino acid to Ser415 and/or Thr613 need not be at the same position as Ser415 (that is, 415 amino acids from the N-terminal of a transferrin protein) or Thr613 (that is, 613 amino acids from the N-terminal of a transferrin protein) in order to be equivalent. For example, the skilled person will readily be able to determine the position of the equivalents of Ser415 and Thr613 within an N-terminally truncated version of SEQ ID No. 1 by a simple alignment of the sequences of SEQ ID No. 1 and the truncated version. Equivalence, in this context, is functional equivalence, such that an amino acid within a transferrin molecule can be said to be equivalent to Ser415 of SEQ ID No. 1 if it is the third amino acid within an N-linked glycosylation site (the first being Asn) of a transferrin protein and is in the closest glycosylation site to the N-terminus of the transferrin protein. Likewise, an amino acid within a transferrin molecule can be said to be equivalent to Thr613 of SEQ ID No. 1 if it is the third amino acid within an N-linked glycosylation site (the first being Asn) of a transferrin protein and is in the second closest glycosylation site to the N-terminus of the transferrin protein.

Equivalents to Asn413 of SEQ ID No. 1, Lys414 of SEQ ID No. 1, Asn611 of SEQ ID No. 1, and Val612 of SEQ ID No. 1 may also be readily determined using the same approach. Equivalents of Asn413 and Asn611 will always be Asn and will be found two amino adds (in the N-terminal direction) away from the equivalents of Ser415 and Thr613, respectively. An equivalent of Lys414 may be any amino acid that is found flanked at either side by equivalents of Asn413 and Ser415. An equivalent of Val612 may be any amino acid that is found flanked at either side by equivalents of Asn611 and Thr615.

Thus, a transferrin protein according to the present invention may differ from the sequence of SEQ ID No. 1, at positions other than those modification already defined by the first, second and third aspects of the invention, by sequence insertions, deletions and substitutions. Accordingly, a transferrin protein can be any members of the transferrin family (Testa, Proteins of iron metabolism, CRC Press, 2002; Harris & Aisen, Iron carriers and iron proteins, Vol. 5, Physical Bioinorganic Chemistry, VCH, 1991) and their derivatives, such as transferrin, mutant transferrins (Mason et al., 1993, Biochemistry, 32, 5472; Mason et al., 1998, Biochem. J., 330(1), 35), truncated transferrins, transferrin lobes (Mason et al, 1996, Protein Expr. Purif., 8, 119; Mason et al., 1991, Protein Expr. Purif., 2, 214), lactoferrin, mutant lactoferrins, trunsated lactoferrins, lactoferrin lobes or fusions of any of the above to other peptides, polypeptides or proteins (Shin et al., 1995, Proc. Natl. Acad. Sci. USA, 92, 2820; All et al, 1999, J. Biol. Chem., 274, 24066; Mason et al, 2002, Biochemistry, 41, 9448), so long as the transferrin protein contains equivalents of the amino acids Asn413, Lys414, Ser415, Asn611, Val612 and Thr613 of SEQ ID No. 1.

The transferrin mutants of the invention may optionally be fused to another protein, particularly a bioactive protein such as those described below. The fusion may be at the N- or C-terminal or comprise insertions. The skilled person will also appreciate that the open reading frame may encode a protein comprising any sequence, be it a natural protein (including a zymogen), or a variant, or a fragment (which may, for example be a domain) of a natural protein; or a totally synthetically protein; or a single or multiple fusion of different proteins (natural or synthetic). Examples of transferrin fusions are given in US patent applications published as US2003-026778, US2003-0221201 and US 2003.0226155, in Shin et al. (1995) Proc. Natl. Acad. Sci. USA. 92m 2820, All et al. (1999) J Biol Chem 274, 24066, Mason et al. 2002, Biochemistry 41, 9448, the content of which are incorporated herein by reference.

The transferrin mutant of the invention may optionally be incorporated in nanobodies using method known within the art such as disclosed in WO 2008/007146.

The transferrin may or may not be human transferrin. The term “human transferrin” is used herein to denote material which is indistinguishable from transferrin derived from a human or which is a variant or fragment thereof. A “variant” includes insertions, deletions and substitutions, either conservative or non-conservative.

Mutants of transferrin are included in the invention. Such mutants may or may not have altered immunogenicity. Transferrin mutants may or may not be altered in their natural binding to metal ions and/or other proteins, such as the transferrin receptor.

We also include naturally-occurring polymorphic variants of human transferrin or human transferrin analogues.

In one embodiment, a transferrin protein, as defined by the first, second or third aspects of the invention, will have a sequence that possesses at least 10%, 20%, 30%, 40%, 50%, 60%, 70%, 80%, 90%, 91%, 92%, 93%, 94%, 95%, 96%, 97%, 98%, or 99% sequence identity to the sequence of SEQ ID No. 1. Sequence identity may be calculated using methods well known in the art, such as according to the methodology described in WO 2006/136831.

Generally, variants or fragments of human transferrin will have at least 5%, 10%, 15%, 20%, 30%, 40% or 50% (preferably at least 80%, 90% or 95%) of the ligand binding capacity (for example iron-binding) of a protein having the sequence of SEQ ID No. 1, weight for weight. The iron binding capacity of transferrin or a test sample can be determined as set out below.

A protein comprising the sequence of a transferrin mutant of the present invention comprises a mutation to, at least, Ser415 (or its equivalent) such that it is replaced by an amino acid that does not allow glycosylation at Asn413 (or its equivalent) and/or a mutation to Thr613 (or its equivalent) such that it is replaced by an amino acid that does not allow glycosylation at Asn611 (or its equivalent). By “does not allow glycosylation” we include the meaning that the Asn amino acid within the same glycosylation site as the mutated amino acid (i.e. Asn413 in the context of the mutation of Ser415, and Asn611 in the context of mutation of Thr613) is not detectably subject to N-linked glycosylation when a gene encoding the recombinant protein comprising the sequence of a transferrin mutant is expressed in a S. cerevisiae host strain in accordance with the protocol given in the examples of this application, and wherein the S. cerevisiae host strain that is chosen is capable of performing N-linked glycosylation at Asn413 and Asn611 of a protein consisting of the sequence of SEQ ID No. 1.

A protein may comprise; the sequence of a transferrin mutant of the present invention where in addition to a mutation in Ser415 to an amino acid that does not allow glycosylation at Asn413 and/or a mutation in Thr613 to an amino acid that does not allow glycosylation in Asn 611., at least one further mutation is introduced that reduces O-linked glycosylation of the protein. By “reduces O-linked glycosylation” we include the meaning that the amino acid whereto O-linked glycosylation is connected in the native transferrin molecule is mutated to an amino acid that can not be glycosylated or a mutation to an amino acid in the context of such an amino acid that results in a lower degree of O-linked glycosylation than is observed in the native transferring molecule. A preferred position for such a mutation is position 32 in SEQ ID NO: 1, more preferred S32A or S32C.

In one embodiment, the mutation(s) made to the transferrin mutant sequence of the invention in order to prevent glycosylation of the mutant does not substantially reduce the biological function of the transferrin mutant. This is assessed in comparison to a “control” protein that possess the same sequence as the recombinant protein comprising the sequence of a transferrin mutant in question, other than for the mutations made to any of Ser32 of SEQ ID NO: 1; Ser415 of SEQ ID No. 1; Asn611 of SEQ ID No. 1; Val612 of SEQ ID No. 1; Thr613 of SEQ ID No. 1; Asn413 of SEQ ID No. 1; Lys414 of SEQ ID No. 1 (or their equivalents) in order to prevent glycosylation, optionally wherein the recombinant protein in question and its control are expressed in the same expression system and isolated using the same method.

The biological function of the mutant, in comparison to the control, refers to at least one, or more, of the iron binding capacity, receptor binding capacity, iron-uptake capacity, and cell culture performance.

Iron binding capacity refers to the ability a recombinant protein comprising the sequence of a transferrin mutant to reversibly bind iron. Thus, in one embodiment, the mutations made to the transferrin sequence in a recombinant protein comprising the sequence of a transferrin mutant of the invention in order to prevent glycosylation of the mutant are considered to not substantially reduce the biological function of the mutant if the mutant possesses at least 50%, 60%, 70%, 80%, 90%, 91%, 92%, 93%, 94%, 95%, 96%, 97%, 98%, 99% or substantially 100% of the iron binding capacity of the control transferrin (and, optionally, no more than 150%, 140%, 130%, 120%, 110%, 105%, 104%, 103%, 102%, 101% or substantially 100% of the iron binding capacity of the control transferrin). Iron binding capacity can be determined spectrophotometrically by 470 nm:280 nm absorbance ratios for the proteins in their iron-free and fully iron-loaded states. Reagents should be iron-free unless stated otherwise. Iron can be removed from transferrin or the test sample by dialysis against 0.1M citrate, 0.1M acetate, 10 mM EDTA pH4.5. Protein should be at approximately 20 mg/mL in 100 mM HEPES, 10 mM NaHCO₃ pH8.0. Measure the 470 nm:280 nm absorbance ratio of apo-transferrin (i.e. iron-free control transferrin) (Calbiochem, CN Biosciences, Nottingham, UK) diluted in water so that absorbance at 280 nm can be accurately determined spectrophotometrically (0% iron binding). Prepare 20 mM iron-nitrilotriacetate (FeNTA) solution by dissolving 191 mg nitrotriacetic acid in 2 mL 1M NaOH, then add 2 mL 0.5M ferric chloride. Dilute to 50 mL with deionised water. Fully load apo-(control) transferrin with iron (100% iron binding) by adding a sufficient excess of freshly prepared 20 mM FeNTA, then dialyse the holo-transferrin preparation completely against 100 mM HEPES, 10 mM NaHCO₃ pH8.0 to remove remaining FeNTA before measuring the absorbance ratio at 470 nm:280 nm. Repeat the procedure using test sample (i.e. the recombinant protein comprising the sequence of a transferrin mutant in question), which should initially be free from iron, and compare final ratios to the control.

In another embodiment, the mutations made to the transferrin sequence in a recombinant protein comprising the sequence of a transferrin mutant of the invention in order to prevent glycosylation of the mutant are considered to not substantially reduce the biological function of the mutant if the mutant possesses at least 50%, 60%, 70%, 80%, 90%, 91%, 92%, 93%, 94%, 95%, 96%, 97%, 98%, 99% or substantially 100% of the receptor binding capacity of the control transferrin (and, optionally, no more than 150%, 140%, 130%, 120%, 110%, 105%, 104%, 103%, 102%, 101% or substantially 100% of the receptor binding capacity of the control transferrin). Receptor binding capacity can be determined by a label-free surface plasmon resonance (SPR) based technology for studying biomolecular interactions in real time, or by radiolabelled iron-uptake assays (see below).

Iron-uptake capacity refers to the ability of a recombinant protein comprising the sequence of a transferrin mutant to bind iron, and bind to the transferrin receptor, and then to be internalised by a cell through receptor-mediated endocytosis, in order to deliver iron into the cell. Thus, in another embodiment, the mutations made to the transferrin sequence in a recombinant protein comprising the sequence of a transferrin mutant of the invention in order to prevent glycosylation of the mutant are considered to not substantially reduce the biological function of the mutant if the mutant possesses at least 50%, 60%, 70%, 80%, 90%, 91%, 92%, 93%, 94%, 95%, 96%, 97%, 98%, 99% or substantially 100% of the iron-uptake capacity of the control transferrin (and, optionally, no more than 150%, 140%, 130%, 120%, 110%, 105%, 104%, 103%, 102%, 101% or substantially 100% of the iron-uptake capacity of the control transferrin). Iron-uptake capacity can be determined by receptor-mediated delivery of radiolabelled transferrin in a ⁵⁵Fe uptake assay using human erythroleukemic K562 cells. This erythroleukemic cell line was the standard in the development of the model of receptor-mediated endocytosis of transferrin and iron donation by this pathway (Klausner et al, 1983, J. Biol. Chem., 258, 4715-4724; Bates & Schlabach, 1973, J. Biol. Chem., 248, 3228-3232). Alternatively, transferrin samples can be compared to each other in a competition assay, for example, where two unlabelled recombinant transferrins are compared for their ability to inhibit radiolabelled iron uptake by a plasma transferrin control.

For iron-55 uptake from labeled diferric transferrin, K562 erythroleukemic cells, cultured in RPMI cell culture medium under standard conditions (bicarbonate-buffered, 5% CO₂, antibiotics, 10% fetal calf serum) is washed with serum-free medium containing HEPES-buffer and 1 mg/ml of bovine serum albumin and used at a concentration of 10 million cells/ml in this medium. The samples tested should be prepared as equimolar concentrations of apo-transferrin. Transferrin can be loaded with iron according to a standard procedure using ferric nitrilotriacetate as iron source. Increasing concentrations of control protein or the respective test protein sample (0, 25, 100, 200, 400, 800, 1600 nM), labeled with ⁵⁵Fe, should be mixed with 25 μl of medium, and the reaction started by the addition of 300 μl of cell suspension. A second series of parallel experiments should be carried out in the presence of a hundredfold excess of unlabeled diferric transferrin to account for unspecific binding. After 25 minutes at 37° C. the reaction should be stopped by immersion into an ice-bath, three aliquots of 60 μl of cell suspension transferred to new tubes and the cells centrifuged in the cold and again after addition of an oil layer of diethylphthalate/dibutylphthalate. The supernatant should be removed, the cell pellet transferred into a counter vial and lysed with 0.5 M KOH+1% Triton X-100. The lysates should be neutralized with 1M HCl after overnight lysis, and mixed with Readysolv scintillation cocktail and counted in the Packard Liquid Scintillation Counter. The results can be presented as fmol ⁵⁵Fe/million cells, and can be used to calculate the dissociation constant (K_{d}) for the transferrin receptor.

For the competition experiments, increasing concentrations of control diferric protein and test diferric protein sample (0, 25, 100, 200, 400, 800, 1600 nM) can be mixed with 100 nM of native diferric plasma transferrin labeled with ⁵⁵Fe in 25 μl of medium. The reaction is started by the addition of 300 μl of cell suspension. After 25 min at 37° C. the reaction is stopped by immersion into an ice-bath, three aliquots of 60 μl of cell suspension are transferred to new tubes and the cells are centrifuged in the cold and again after addition of an oil layer of diethylphtalate/dibutylphthalate. The supernatant is removed, the cell pellet transferred into a counter vial and lysed with 0.5 M KOH+1% Triton X-100. The lysates are neutralized with 1N HCl after overnight lysis, mixed with Readysolv scintillation cocktail and counted in the Packard Liquid Scintillation Counter.

In another embodiment, the mutations made to the transferrin sequence in a recombinant protein comprising the sequence of a transferrin mutant of the invention in order to prevent glycosylation of the mutant are considered to not substantially reduce the biological function of the mutant if the mutant possesses at least 50%, 60%, 70%, 80%, 90%, 91%, 92%, 93%, 94%, 95%, 96%, 97%, 98%, 99% or substantially 100% of cell culture performance of the control transferrin (and, optionally, no more than 150%, 140%, 130%, 120%, 110%, 105%, 104%, 103%, 102%, 101% or substantially 100% of the cell culture performance of the control transferrin). Cell culture performance can be determined by the method described by Keenan et al, 2006, Cytotechnology, 51, 29-37, the methodology of which is incorporated herein by reference.

As used herein, the term “conservative” amino acid substitutions refers to substitutions made is within the same group, and which typically do not substantially affect protein function. In one embodiment, the following diagram may be used to determine conservative amino acid substitutions—

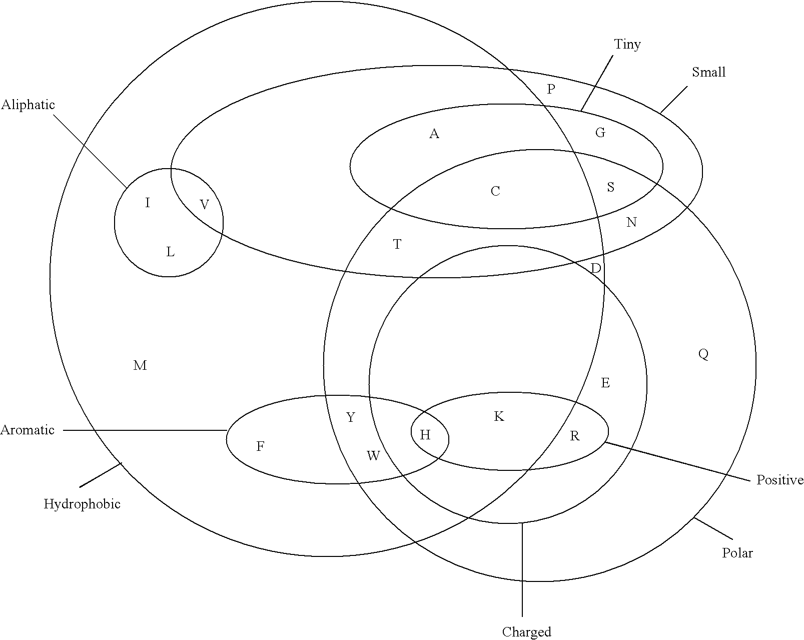

In another embodiment, “conservative” amino acid substitutions refers to substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

Accordingly, for example, a conservative substitution of Ser415 can include glycine or alanine. A conservative substitution of Thr613 can include glycine, alanine, valine or methionine. A conservative substitution of Asn 413 and/or Asn611 can include glutamine and aspartic acid.

Non-conservative substitutions encompass substitutions of amino acids in one group by amino acids in another group. For example, a non-conservative substitution could include the substitution of a polar amino acid for a hydrophobic amino acid.

A polynucleotide (such as a DNA or RNA molecule) may be produced, comprising a sequence that encodes a protein comprising the sequence of a transferrin mutant as defined above by any one of the first, second or third aspects of the present invention. It may be a gene that encodes a protein comprising the sequence of a recombinant transferrin mutant.

A gene encoding a protein comprising the sequence of a transferrin mutant comprises a polynucleotide sequence encoding the protein comprising the sequence of a transferrin mutant (typically according to standard codon usage for any given organism), designated the open reading frame (“ORF”). The gene may additionally comprise some polynucleotide sequence that does not encode an open reading frame (termed “non-coding region”).

Non-coding regions in the gene may contain one or more regulatory sequences, operatively linked to the ORF, which allow for the transcription of the open reading frame and/or translation of the resultant transcript.

The term “regulatory sequence” refers to a sequence that modulates (i.e., promotes or reduces) the expression (i.e., the transcription and/or translation) of an ORF to which it is operably linked. Regulatory regions typically include promoters, terminators, ribosome binding sites and the like. The skilled person will appreciate that the choice of regulatory region will depend upon the intended expression system. For example, promoters may be constitutive or inducible and may be cell- or tissue-type specific or non-specific.

Suitable regulatory regions, may be 5 bp, 10 bp, 15 bp, 20 bp, 25 bp, 30 bp, 35 bp, 40 bp, 45 bp, 50 bp, 60 bp, 70 bp, 80 bp, 90 bp, 100 bp, 120 bp, 140 bp, 160 bp, 180 bp, 200 bp, 220 bp, 240 bp, 260 bp, 280 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1100 bp, 1200 bp, 1300 bp, 1400 bp, 1500 bp or greater, in length.

Those skilled in the art will recognise that the gene encoding the recombinant protein comprising the sequence of a transferrin mutant may additionally comprise non-coding regions and/or regulatory regions. Such non-coding regions and regulatory regions are not restricted to the native non-coding regions and/or regulatory regions normally associated with the chaperone ORF.

Where the expression system (i.e. the host cell) is yeast, such as Saccharomyces cerevisiae, suitable promoters for S. cerevisiae include those associated with the PGK1 gene, GAL1 or GAL10 genes, TEF1, TEF2, PYK1, PMA1, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, α-mating factor pheromone, a-mating factor pheromone, the PRB1 promoter, the PRA1 promoter, the GPD1 promoter, and hybrid promoters involving hybrids of parts of 5′ regulatory regions with parts of 5′ regulatory regions of other promoters or with upstream activation sites (e.g. the promoter of EP-A-258 067).

Suitable transcription termination signals are well known in the art. Where the host cell is eukaryotic, the transcription termination signal is preferably derived from the 3′ flanking sequence of a eukaryotic gene, which contains proper signals for transcription termination and polyadenylation. Suitable 3′ flanking sequences may, for example, be those of the gene naturally linked to the expression control sequence used, i.e. may correspond to the promoter. Alternatively, they may be different. In that case, and where the host is a yeast, preferably S. cerevisiae, then the termination signal of the S. cerevisiae ADH1, ADH2, CYC1, or PGK1 genes are preferred.

It may be beneficial for the promoter and open reading frame of the gene encoding the recombinant protein comprising the sequence of a transferrin mutant to be flanked by transcription termination sequences so that the transcription termination sequences are located both upstream and downstream of the promoter and open reading frame, in order to prevent transcriptional read-through into any neighbouring genes, such as 2 μm genes, and vice versa.

In one embodiment, the favoured regulatory sequences in yeast, such as Saccharomyces cerevisiae, include: a yeast promoter (e.g. the Saccharomyces cerevisiae PRB1 promoter), as taught in EP 431 880; and a transcription terminator, preferably the terminator from Saccharomyces ADH1, as taught in EP 60 057.

It may be beneficial for the non-coding region to incorporate more than one DNA sequence encoding a translational stop codon, such as UAA, UAG or UGA, in order to minimise translational read-through and thus avoid the production of elongated, non-natural fusion proteins. The translation stop codon UM is preferred.

The term “operably linked” includes within its meaning that a regulatory sequence is positioned within any non-coding region in a gene such that it forms a relationship with an ORF that permits the regulatory region to exert an effect on the ORF in its intended manner. Thus a regulatory region “operably linked” to an ORF is positioned in such a way that the regulatory region is able to influence transcription and/or translation of the ORF in the intended manner, under conditions compatible with the regulatory sequence.

In one preferred embodiment, the recombinant protein comprising the sequence of a transmutant is secreted. In that case, a sequence encoding a secretion leader sequence may be included in the open reading frame. Thus, a polynucleotide according to the fourth aspect of the present invention may comprise a sequence that encodes a recombinant protein comprising the sequence of a transferrin mutant operably linked to a polynucleotide quence that encodes a secretion leader sequence. Leader sequences are usually, although not necessarily, located at the N-terminus of the primary translation product of an ORF and are generally, although not necessarily, cleaved off the protein during the secretion process, to yield the “mature” protein. Thus, in one embodiment, the term “operably linked” in the context of leader sequences includes the meaning that the sequence that encodes a recombinant protein comprising the sequence of a transferrin mutant is linked, at its 5′ end, and in-frame, to the 3′ end of a polynucleotide sequence that encodes a secretion leader sequence. Alternatively, the polynucleotide sequence that encodes a secretion leader sequence may be located, in-frame, within the coding sequence of the recombinant protein comprising the sequence of a transferrin mutant, or at the 3′ end of the coding sequence of the recombinant protein comprising the sequence of a transferrin mutant.

Numerous natural or artificial polypeptide leader sequences (also called secretion pre regions and pre/pro regions) have been used or developed for secreting proteins from host cells. Leader sequences direct a nascent protein towards the machinery of the cell that exports proteins from the cell into the surrounding medium or, in some cases, into the periplasmic space.

For production of proteins in eukaryotic species such as the yeasts Saccharomyces cerevisiae, Zygosaccharomyces species, Kluyveromyces lactis and Pichia pastoris, known leader sequences include those from the S. cerevisiae acid phosphatase protein (Pho5p) (see EP 366 400), the invertase protein (Suc2p) (see Smith et al., (1985) Science, 229, 1219-1224) and heat-shock protein-150 (Hsp150p) (see WO 95/33833). Additionally, leader sequences from the S. cerevisiae mating factor alpha-1 protein (MFα-1) and from the human lysozyme and human serum albumin (HSA) protein have been used, the latter having been used especially, although not exclusively, for secreting human albumin. WO 90/01063 discloses a fusion of the MFα-1 and HSA leader sequences. In addition, the natural transferrin leader sequence may or may not be used to direct secretion of the recombinant protein comprising the sequence of a transferrin mutant.

Polynucleotides according to the fifth aspect of the present invention may be integrated into a plasmid, according to the sixth aspect of the present invention. The skilled person will appreciate that any suitable plasmid may be used, such as a centromeric plasmid. Other suitable plasmids include a yeast-compatible 2 μm-based plasmid. WO 2005/061718 provides a full description of suitable plasmids, the contents of which are incorporated herein by reference. Furthermore, as also disclosed in WO 2005/061718, the plasmid may comprise a gene encoding a chaperone, such as protein disulphide isomerase (PDI), for co-expression with the plasmid-encoded gene for the protein comprising the sequence of a transferrin mutant.

Polynucleotides or plasmids according to the fifth and sixth aspects of the present invention can be used to transform a host cell. The host cell may be any type of cell. The host cell may or may not be an animal (such as mammalian, avian, insect, etc.), plant, fungal or bacterial cell. Bacterial and fungal, such as yeast, host cells may or may not be preferred.

In one embodiment the host cell is a yeast cell, such as a member of the Saccharomyces, Kluyveromyces, or Pichia genus, such as Saccharomyces cerevisiae, Kluyveromyces lactis, Pichia pastoris and Pichia membranaefaciens, or Zygosaccharomyces rouxii, Zygosaccharomyces bailii, Zygosaccharomyces fermentati, Hansenula polymorpha (also known as Pichia angusta) or Kluyveromyces drosophilarum are preferred.

In one further embodiment the host cell is a fungal cell, such as Aspergillus niger, Aspergillus oryzae, Trichoderma, Fusarium venenatum, Pichia angusta or Hansenula polymorpha.

It may be particularly advantageous to use a host cell, such as a yeast host cell, that is deficient in one or more protein mannosyl transferases involved in O-glycosylation of proteins, for instance by disruption of the gene coding sequence. WO 94/04687 discloses yeast strains deficient in one or more of the PMT genes and this is discussed further in WO 2005/061718, the contents of which are incorporated herein by reference. Alternatively, the yeast could be cultured in the presence of a compound that inhibits the activity of one of the PMT genes (Duffy et al, “Inhibition of protein mannosyltransferase 1 (PMT1) activity in the pathogenic yeast Candida albicans”, International Conference on Molecular Mechanisms of Fungal Cell Wall Biogenesis, 26-31 Aug. 2001, Monte Verita, Switzerland, Poster Abstract P38; the poster abstract may be viewed at http://www.micro.biol.ethz.ch/cellwall/).

In one embodiment, the host cell may over-express a chaperone, such as PDI or another chaperone as discussed in WO 2005/061718, WO 2006/067511 or WO 2006/136831, the contents of which are each incorporated herein by reference. For example, the host cell may comprise one or more additional chromosomal copies of a chaperone (e.g. PDI) gene, in addition to its endogenous copy or may, for example, be genetically modified to cause overexpression of its endogenous chaperone (e.g. PDI) gene.

Suitable methods for transformation of animal cells are well known in the art and include, for example the use of retrovirus vectors (such as lentivirus vectors). Wolkowicz et al, 2004, Methods Mol. Biol., 246, 391-411 describes the use of lentivirus vectors for delivery of recombinant nucleic acid sequences to mammalian cells for use in cell culture techniques. Fassler, 2004, EMBO Rep., 5(1), 28-9 reviews lentiviral transgene vectors and their use in the production of transgenic systems. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

With regard to transformation of prokaryotic host cells, see, for example, Cohen et al (1972) Proc. Natl. Acad. Sci. USA 69, 2110 and Sambrook et al (2001) Molecular Cloning, A Laboratory Manual, 3^{rd} Ed. Cold Spring Harbor Laboratory, Cold Spring Harbor, N.Y.

Transformation of yeast cells is described in Sherman et al (1986) Methods In Yeast Genetics, A Laboratory Manual, Cold Spring Harbor, N.Y. The method of Beggs (1978) Nature 275, 104-109 is also useful. Methods for the transformation of S. cerevisiae are taught generally in EP 251 744, EP 258 067 and WO 90/01063, all of which are incorporated herein by reference.

Electroporation is also useful for transforming cells and is well known in the art for transforming fungal (including yeast) cell, plant cells, bacterial cells and animal (including vertebrate) cells. Methods for transformation of yeast by electroporation are disclosed in Becker & Guarente (1990) Methods Enzymol. 194, 182.

A polynucleotide or plasmid as defined above, may be introduced into a host through the above-mentioned standard techniques. Generally, the polynucleotide or plasmid will transform not all of the hosts and it will therefore be necessary to select for transformed host cells. Thus, a polynucleotide or plasmid may comprise a selectable marker, including but not limited to bacterial selectable marker and/or a yeast selectable marker. A typical bacterial selectable marker is the β-lactamase gene although many others are known in the art. Typical yeast selectable marker include LEU2, TRP1, HIS3, HIS4, URA3, URA5, SFAf, ADE2, MET15, LYS5, LYS2, ILV2, FBA1, PSE1, PDI1 and PGK1.

One selection technique involves incorporating into the polynucleotide or plasmid a DNA sequence marker, with any necessary control elements, that codes for a selectable trait in the transformed cell. These markers include dihydrofolate reductase, G418, neomycin or zeocin resistance for eukaryotic cell culture, and tetracycline, kanamycin, ampicillin (i.e. β-lactamase) or zeocin resistance genes for culturing in E. coli and other bacteria. Zeocin resistance vectors are available from Invitrogen. Alternatively, the gene for such selectable trait can be on another vector, which is used to co-transform the desired host cell.

Another method of identifying successfully transformed cells involves growing the cells resulting from the introduction of a polynucleotide or plasmid, optionally to allow the expression of a recombinant polypeptide (i.e. a polypeptide which is encoded by a polynucleotide sequence on the plasmid and is heterologous to the host cell, in the sense that that polypeptide is not naturally produced by the host). The recombinant polypeptide may or may not be the recombinant protein comprising the sequence of a transferrin mutant of the invention. Cells can be harvested and lysed and their DNA or RNA content examined for, the presence of the recombinant sequence using a method such as that described by Southern (1975) J. Mol. Biol. 98, 503 or Berent et al (1985) Biotech. 3, 208 or other methods of DNA and RNA analysis common in the art. Alternatively, the presence of a polypeptide in the supernatant of a culture of a transformed cell can be detected using antibodies.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Following selection of a transformed host cell, it can be cultured under conditions that allow for the expression of the recombinant protein comprising the sequence of a transferrin mutant. Appropriate conditions known to those skilled in the art, and in view of the teachings disclosed herein. The culture medium may be non-selective or place a selective pressure on the host cell's maintenance of a polypeptide or plasmid of the fourth or fifth aspects of the present invention.

The thus produced recombinant protein comprising the sequence of a transferrin mutant may be present intracellularly or, if secreted, in the culture medium and/or periplasmic space of the host cell. It may therefore be appropriate to perform the further step of isolating the expressed recombinant protein from the cultured host cell, recombinant organism or culture medium.

The step of “of isolating the expressed recombinant protein from the cultured host cell, recombinant organism or culture medium” optionally comprises cell immobilisation, cell separation and/or cell breakage, but always comprises at least one other purification step different from the step or steps of cell immobilisation, separation and/or breakage.

Cell immobilisation techniques, such as encasing the cells using calcium alginate bead, are well known in the art. Similarly, cell separation techniques, such as centrifugation, filtration (e.g. cross-flow filtration, expanded bed chromatography and the like) are well known in the art. Likewise, methods of cell breakage, including beadmilling, sonication, enzymatic exposure and the like are well known in the art.

Techniques known in the art can be employed to recover the expressed recombinant protein.

In one embodiment, the expressed recombinant protein comprising the sequence of a transferrin mutant is secreted by the host, and recovered from the cell culture medium by centrifugation and collection of the supernatant to yield a partially purified recombinant protein.

The partially purified recombinant protein may be further purified from the supernatant by one or more art-known protein purification steps. Methods for purifying transferrin are disclosed, for example, in U.S. Pat. No. 5,986,067; U.S. Pat. No. 6,251,860; U.S. Pat. No. 5,744,586; and U.S. Pat. No. 5,041,537. Although some of these documents refer to purification of transferrin from plasma, rather than from a recombinant host cell, some of the steps used therein may, nevertheless, be usefully applied. Furthermore, any known technique that has been found to be useful for purifying proteins may be used. Suitable methods include ammonium sulphate or ethanol precipitation, acid or solvent extraction, anion or cation exchange chromatography, phosphocellulose chromatography, hydrophobic interaction chromatography, affinity chromatography, hydroxyapatite chromatography, lectin chromatography, concentration, dilution, pH adjustment, diafiltration, ultrafiltration, high performance liquid chromatography (“HPLC”), reverse phase HPLC, conductivity adjustment and the like. For example, in one embodiment, one or more ion exchange steps may be used. For example, a cation exchange step that is run in the positive or negative mode with respect to the recombinant protein comprising the sequence of a transferrin mutant may be used, optionally followed (with or without intervening purification steps) by an anion exchange step that is run in the positive or negative mode with respect to the recombinant protein comprising the sequence of a transferrin mutant, or vice versa.

The thus isolated recombinant protein comprising the sequence of a transferrin mutant may be provided in iron-free (i.e. “apo”) form as a recombinant protein comprising the sequence of an apo-transferrin mutant, or may be subjected to holoization (i.e. saturation with Fe³⁺ ions) using art-known techniques to produce a recombinant protein comprising the sequence of a holo-transferrin mutant. The finally produced preparation of recombinant protein comprising the sequence of a transferrin mutant may be partially or fully holoized. For example, it may possess an iron binding capacity of less than 99%, 95%, 90%, 80%, 70%, 60%, 50% 40%, 30%, 20%, 15%, 14%, 13%, 12%, 11%, 10%, 9%, 8%, 7%, 6%, 5%, 4%, 3%, 2%, 1% or substantially 0%. Iron binding capacity may be determined, for example, by the method of EP 1 094 835 81 (see paragraphs 49-51, the contents of which are incorporated herein by reference).

The isolated recombinant protein comprising the sequence of a transferrin mutant may be further manipulated to modify its concentration or environment, for example using art-known techniques such as ultrafiltration and diafiltration. In one embodiment, the isolated recombinant protein comprising the sequence of a transferrin mutant may be provided at a concentration of about 10⁻⁴ g.L⁻¹, 10⁻³ g.L⁻¹, 0.01 g.L⁻¹, 0.02 g.L⁻¹, 0.03 g.L⁻¹, 0.04 g.L⁻¹, 0.05 g.L⁻¹, 0.06 g.L⁻¹, 0.07 g.L⁻¹, 0.08 g.L⁻¹, 0.09 g.L⁻¹, 0.1 g.L⁻¹, 0.2 g.L⁻¹, 0.3 g.L⁻¹, 0.4 g.L⁻¹, 0.5 g.L⁻¹, 0.6 g.L⁻¹, 0.7 g.L⁻¹, 0.8 g.L⁻¹, 0.9 g.L⁻¹, 1 g.L⁻¹, 2 g.L⁻¹, 3 g.L⁻¹, 4 g.L⁻¹, 5 g.L⁻¹, 6 g.L⁻¹, 7 g.L⁻¹, 8 g.L⁻¹, 9 g.L⁻¹, 10 g.L⁻¹, 15 g.L⁻¹, 20 g.L⁻¹, 25 g.L⁻¹, 30 g.L⁻¹, 40 g.L⁻¹, 50 g.L⁻¹, 60 g.L⁻¹, 70 g.L⁻¹, 70 g.L⁻¹, 90 g.L⁻¹, 100 g.L⁻¹, 150 g.L⁻¹, 200 g.L⁻¹, 250 g.L⁻¹, 300 g.L⁻¹, 350 g.L⁻¹, 400 g.L⁻¹, 500 g.L⁻¹, 600 g.L⁻¹, 700 g.L⁻¹, 800 g.L⁻¹, 900 g.L⁻¹, 1000 g.L⁻¹, or more. A concentration of 1-100 g.L⁻¹, such as 10-50 g.L⁻¹, 15-25 g.L⁻¹, or 18-22 g.L⁻¹, for example, approximately 20 g.L⁻¹ may be preferred.

The isolated recombinant protein comprising the sequence of a transferrin mutant may also be subjected to sterilisation using art known techniques, such as 0.22 μm filtration.

A commercially or industrially acceptable level of purity may be obtained by a relatively crude purification method by which the recombinant protein comprising the sequence of a transferrin mutant is put into a form suitable for its intended purpose. A protein preparation that has been purified to a commercially or industrially acceptable level of purity may, in addition to the recombinant protein comprising the sequence of a transferrin mutant, also comprise, for example, cell culture components such as host cells or debris derived therefrom. Alternatively, high molecular weight components (such as host cells or debris derived therefrom) may or may not be removed (such as by filtration or centrifugation) to obtain a composition comprising the recombinant protein comprising the sequence of a transferrin mutant and, optionally, a functionally acceptable level of low molecular weight contaminants derived from the cell culture process.

The isolated recombinant protein comprising the sequence of a transferrin mutant may or may not be purified to achieve a pharmaceutically acceptable level of purity. A protein has a pharmaceutically acceptable level of purity if it is essentially pyrogen free and can be administered in a pharmaceutically efficacious amount without causing medical effects not associated with the activity of the protein.

The resulting isolated recombinant protein comprising the sequence of a transferrin mutant may be used for any of its known utilities, which includes i.v. administration to patients to treat various conditions, and supplementing culture media, and as an excipient in formulations of other proteins.

The isolated recombinant protein of the invention may be formulated into pharmaceutical compositions using methods well known within the art and administered for the treatment of indications known to the treatable by transferrin, such as plasma transferrin 1. As an example of a known clinical use of transferrin can be found in the U.S. patent application Ser. No. 10/405,612.

The isolated recombinant protein of the invention may also be used for applications having a known use of transferrin such as general medical uses, coatings and biomaterials. As an example of biomaterials wherein the proteins of the invention may be used can be mentioned Ghosh et al (2008) Angew. Chem. Int. Ed 47, 2217-2221.

A method of the present invention may or may not further comprise the step of formulating the isolated recombinant protein comprising the sequence of a transferrin mutant with a carner or diluent and optionally presenting the thus formulated protein in a unit dosage form.

Although it is possible for an isolated recombinant protein comprising the sequence of a transferrin mutant obtained by a process of the invention to be administered alone, it is preferable to present it as a pharmaceutical formulation, together with one or more acceptable carriers or diluents. The carrier(s) or diluent(s) must be “acceptable” in the sense of being compatible with the desired protein and not deleterious to the recipients thereof. Typically, the carriers or diluents will be water or saline which will be sterile and pyrogen free.

Optionally the thus formulated recombinant protein comprising the sequence of a transferrin mutant will be presented in a unit dosage form, such as in the form of a tablet, capsule, injectable solution or the like.

Alternatively, a method of the present invention may or may not further comprise the step of lyophilising the thus isolated recombinant protein comprising the sequence of a transferrin mutant.

As discussed above, a tenth aspect of the present invention provides a mammalian cell culture medium comprising a recombinant protein comprising the sequence of a transferrin mutant according to any one of the first, second, third or fourth aspects of the invention and one or more components selected from the group consisting of; glutamine, insulin, albumin, ethanolamine, fetuin, vitamins, lipoprotein, fatty acids, amino acids, sodium selenite, peptone, insulin-like growth factors and antioxidants.

In one embodiment of the tenth aspect of the present invention the composition comprises (i) basal media; (ii) a recombinant protein comprising the sequence of a transferrin mutant; and one or more components selected from the group consisting of insulin, sodium selenite, glutamine, albumin, peptone, ethanolamine, fetuin, vitamins, lipoprotein, fatty acids insulin-like growth factors and amino acids.

The composition may comprise, for example, between 0.0001-10%, 0.0005-7.5%, 0.001-5.0%, most particularly between 0.05-3.0% (w/v) recombinant protein comprising the sequence of a transferrin mutant according to the present invention.

The composition may comprise between 0.001-1000 mg/L, more particular between 0.01-500 mg/L, even more particular between 0.01-100 mg/L and most particular between 0.04-10 mg/L albumin. The albumin may be recombinant albumin in which case it is preferably obtained from a serum-free source and is substantially free of any other animal-derived proteins prior to its addition to the composition, for example as disclosed in WO 2000/044772 the contents of which are incorporated herein by reference.

The composition may comprise between 0.01-1000 mg/L, more particular between 0.01-500 mg/L, even more particular between 0.1-100 mg/L, such as 1-50 mg/L and most particular between 4-20 mg/L insulin. The insulin may be recombinant insulin in which case it is preferably obtained from a serum-free source and is substantially free of any other animal-derived proteins prior to its addition to the composition.

The composition may comprise between 0.0001-10 mg/L, more particular between 0.005-7.5 mg/L, even more particular between 0.1-5.0 mg/L and most particular between 0.75-3.5 mg/L lipoprotein. The lipoprotein may be recombinant lipoprotein in which case it is preferably obtained from a serum-free source and is substantially free of any other animal-derived proteins prior to its addition to the composition.

The composition may comprise between 0.00001-50 mg/L IGF, more particular between 0.001-5.0 mg/L, even more particular between 0.01-1.0 mg/L and most particular between 0.04-0.2 mg/L IGF. The IGF may be recombinant IGF in which case it is preferably obtained from a serum-free source and is substantially free of any other animal-derived proteins prior to its addition to the composition.

In another embodiment of the tenth aspect of the present invention, the composition comprises (i) basal media; (ii) recombinant protein comprising the sequence of a transferrin mutant of the invention; (iii) insulin; (iv) sodium selenite; and/or (v) albumin.

The composition may comprises at least 2, 2.5, 3, 3.5, 4, 4.5, 5, 5.5, 6, 6.5, 7, 7.5, 8, 8.5 or 9 mg/ml albumin (optionally recombinant albumin as discussed above); at least 3.0, 3.5, 4.0, 4.5, 5.0, 5.5, 6.0, 7.0, 10, 15 or 20 μg/ml recombinant protein comprising the sequence of a transferrin mutant of the invention; approximately 5.5, 6.0, 6.5, 7.0, 7.5, 8.0, 8.5, 9.0, 9.5, 10, 10.5, 11, 11.5, 12, 15 or 20 μg/ml insulin (optionally recombinant insulin as discussed above); at least 1, 2, 3, 2.5, 3, 3.5, 4, 4.5, 5, 5.5, 6, 6.5, 6.7, 7.0, 7.5, 8.0, 9.0, 10, 15 or 20 μg/L sodium selenite.

In one specific embodiment, a cell culture media may comprise approximately 4 mg/ml albumin; approximately 5.5 μg/ml recombinant protein comprising the sequence of a transferrin mutant of the invention; approximately 10 μg/ml insulin; approximately 6.7 μg/L sodium selenite in basal media.

In another embodiment of the ninth aspect of the present invention, the composition comprises (i) basal media; (ii) albumin (optionally recombinant albumin as discussed above) (iii) glutamine; (iv) insulin (optionally recombinant insulin as discussed above); (v) recombinant protein comprising the sequence of a transferrin mutant of the invention; and/or (vi) ethanolamine. The composition may comprise approximately 1, 2, 3, 4, 5, 6, 7, 8, 9, or 10 mM glutamine; approximately 0.1, 0.2, 0.3, 0.4, 0.5, 0.6, 0.7, 0.8, 0.9, 1, 2, 3, 4, 5, 6, 7, or 8% (w/v) albumin; approximately 1, 2, 3, 4, 5, 6, 7, 8, 9, 9.5, 10, 10.5, 11, 11.5, 12, 13, 14, 15, 16, 17, 18, 19, 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L recombinant protein comprising the sequence of a transferrin mutant of the invention; and approximately 1, 2, 3, 4, 5, 6, 7, 8, 8.5, 9, 9.5, 10, 10.5, 11, 11.5, 12, 13, 14, 15, 16, 17, 18, 19 or 20 μM ethanolamine in basal media.

In one specific embodiment, a cell culture media may comprise approximately 4 mM glutamine; approximately 0.5% albumin; approximately 10 mg/L insulin; approximately 1 mg/L recombinant protein comprising the sequence of a transferrin mutant of the invention; and/or approximately 10 μM ethanolamine, in basal media.

In another embodiment of the ninth aspect of the present invention, the composition may include (i) basal media; (ii) albumin (optionally recombinant albumin as discussed above); and one or more of the following components selected from the group consisting of (iii) glutamine; (iv) insulin (optionally recombinant insulin as discussed above); and (v) recombinant protein comprising the sequence of a transferrin mutant of the invention. In one embodiment, the composition comprises approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% (w/v) albumin; approximately 1, 2, 3, 4, 5, 6, 7, 8, 9, 9.5, 10, 10.5, 11, 11.5, 12, 13, 14, 15, 16, 17, 18, 19, or 20 mg/L insulin; and or approximately 0.1, 0.2, 0.3, 0.4, 0.5, 0.6, 0.7, 0.8, 0.9, 0.95, 1, 1.5, 2, 2.5, 3, 4, 5, 6, or 7 mg/L recombinant protein comprising the sequence of a transferrin mutant of the invention. In one specific embodiment, the composition comprises approximately 4 mM glutamine, approximately 1% (w/v) albumin, approximately 10 mg/L insulin, and/or approximately 1 mg/L recombinant protein comprising the sequence of a transferrin mutant of the invention, in basal media.

In another embodiment of the ninth aspect of the present invention, the composition may comprise (i) basal media; (ii) glutamine; (iii) recombinant albumin (optionally recombinant albumin as discussed above); (iv) insulin (optionally recombinant insulin as discussed above); and/or (v) recombinant protein comprising the sequence of a transferrin mutant of the invention; and/or (vii) peptone. In one embodiment, the composition comprises approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% (w/v) albumin; approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L recombinant protein comprising the sequence of a transferrin mutant of the invention; and/or approximately 0.01, 0.02, 0.03, 0.04, 0.05, 0.06, 0.07, 0.08, 0.09, 0.1, 0.2, 0.3, 0.4, 0.5, 1, 2 or 3% (w/v) peptone. In one specific embodiment, the composition may comprise approximately 4 mM glutamine, approximately 1% (w/v) recombinant albumin, approximately 10 mg/L insulin, approximately 1 mg/L recombinant protein comprising the sequence of a transferrin mutant of the invention, and/or approximately 0.1% (w/v) peptone in basal media.

In embodiments of the present invention, the peptone or peptone mixture is a protein hydrolysate, which is obtained from hydrolyzed animal or plant protein. The peptones can be derived from animal by-products from slaughter houses, purified gelatin, or plant material. The protein from the animal or plant sources can be hydrolyzed using acid, heat or various enzyme preparations. Peptone mixtures that can be used include SPY peptone, “Primatone RL” and/or “Primatone HS”, both of which are commercially available (Sheffield, England or; Quest International (IPL:5×59051), PRIMATONE® RL). Alternatively, peptone can be generated from non-animal-derived products, such as plant-derived peptone.

In another embodiment of the ninth aspect of the present invention, the composition may comprise (i) basal media; (ii) glutamine; (iii) albumin (optionally recombinant albumin as discussed above); (iv) insulin (optionally recombinant insulin as discussed above); (v) recombinant protein comprising the sequence of a transferrin mutant of the invention; and/or (vi) fetuin (such as Pedersen). In one embodiment, the composition may comprise approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% (w/v) albumin; approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L recombinant protein comprising the sequence of a transferrin mutant of the invention; and/or approximately 2, 3, 4, 5, 6, 7, 8, 9, 10, 10.5, 11, 11.5, 12, 12.5, 13, 13.5, 14, 14.5, 15, 16, 17, 18, 19, 20 μg/ml of fetuin. In one specific embodiment, the composition of the present invention may comprise approximately 4 mM glutamine, approximately 1% (w/v) albumin, approximately 10 mg/L insulin, approximately 1 mg/L recombinant protein comprising the sequence of a transferrin mutant of the invention, and/or approximately 12.5 μg/ml fetuin (such as Pedersens) in basal media.

In another embodiment of the ninth aspect of the present invention, the composition may comprise (i) basal media; (ii) albumin (optionally recombinant albumin as discussed above) (iii) glutamine; (vi) insulin (optionally recombinant albumin as discussed above); (v) recombinant protein comprising the sequence of a transferrin mutant of the invention; and/or (vi) vitamin E. In one embodiment, the composition may comprise approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% (w/v) albumin; approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L recombinant protein comprising the sequence of a transferrin mutant of the invention; and/or approximately 1, 2, 3, 4, 5, 6, 7, 8, 9, or 10 micromolar vitamin E. In one specific embodiment, the composition of the present invention may comprise approximately 4 mM glutamine, approximately 1% (w/v) recombinant albumin, approximately 10 mg/L insulin, approximately 1 mg/L recombinant protein comprising the sequence of a transferrin mutant of the invention, and/or approximately 5 μM vitamin E in basal media. The composition may comprise approximately 4 mM glutamine, approximately 1% (w/v) recombinant albumin, approximately 10 mg/L insulin, approximately 1 mg/L recombinant protein comprising the sequence of a transferrin mutant of the invention, approximately 0.1% (w/v) peptone, approximately 12.5 μg/mL fetuin (such as Pederson), and/or approximately 5 μM vitamin E.

In a further embodiment, the composition may comprise any media listed in table 1 in WO 2005/070120 hereby incorporated by reference. In another embodiment, the serum free media is either Hybridoma Media, animal component free or Ex-Cell (JRH Biosciences, Inc.).

In another aspect of the present invention compositions are provided that are useful as a cell culture medium that serves to increase the yield of biological products, such as proteins, produced by the cells cultured in the media. In one embodiment, compositions can increase the yield of biological products at least 25%, 30%, 50%, 100%, 200% or 300%. In another embodiment, the biological products produced can be a peptide, such as a therapeutic or diagnostic peptide, polypeptide, protein, monoclonal antibody, immunoglobulin, cytokine (such as interferon), integrin, antigen, growth factor, cell cycle protein, hormone, neurotransmitter, receptor, fusion peptide, blood protein and/or chimeric protein.

The biological products produced may or may not comprise a biological product selected from the list comprising 4-1BB ligand, 5-helix protein, human C-C chemokine, human L105 chemokine, a human L105 chemokine designated huL105_{—}3, monokine induced by gammainterferon (MIG), a partial CXCR4B protein, platelet basic protein (PBP), aiᵢ-antitrypsin, ACRP-30 Homologue; Complement Component C1q C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase 8, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C-C chemokine, MCP2, CCR5 variant, CCR7, CCR7, CD11a Mab, CD137: 4-1BB Receptor Protein, CD20 Mab, CD27, CD27L, CD30, CD30 ligand, CD33 immunotoxin, CD40, CD40L, CD52 Mab, cerebus protein, chemokine eotaxin, chemokine hIL-8, chemokine hMCP1, chemokine hMCP1a, chemokine hMCP1b, chemokine hMCP2, chemokine hMCP3, chemokine hSDF1b, chemokine MCP-4, chemokine TECK and TECK variant, chemokine-like protein IL-8M1 Full-Length and MatLire, chemokine-like protein IL-8M10 Full-Length and Mature, chemokine-like protein IL-8M3, chemokine-like protein IL-8M8 Full-Length and Mature, chemokine-like protein IL-8M9 Full-Length and Mature, chemokine-like protein PF4-414 Full-Length and Mature, chemokine-like protein PF4-426 Full-Length and Mature, chemokine-like protein PF4-M2 Full-Length and Mature, cholera vaccine, chondromodulin-like protein, c-kit ligand; SCF; Mast cell growth factor, MGF; Fibrosarcoma-derived stem cell factor, CNTF and fragment thereof (such as CNTF_{Ax15} (Axokine™), coagulation factors in both pre and active forms, collagens, complement C5 Mab, connective tissue activating protein-III, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_{—}7, DIL-40, Dnase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and, Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; HST-1; HBGF-4, FGF-5, FGF-6; heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, myofibrillar protein Troponin I, FSH, Galactosidase, Galectin-4, G-CSF, GDF-1, Gene therapy, Glioma-derived growth factor, glucagon, glucagon-like peptides, Glucocerebrosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GROgamma), hAPO-4; TROY, hCG, hepatitus B surface Antigen, hepatitus B Vaccine, HER2Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, human Act-2 protein, human adipogenesis inhibitory factor, human B cell stimulating factor-2 receptor, human beta-chemokine H1305 (MCP-2), human C-C chemokine DGWCC, human CC chemokine ELC protein, human CC type chemokine interleukin C, human CCC3 protein, human CCF18 chemokine, human CC-type chemokine protein designated SLC (secondary lymphoid chemokine), human chemokine beta-8 short forms, human chemokine C10, human chemokine CC-2, human chemokine CC-3, human chemokine CCR-2, human chemokine Ckbeta-7, human chemokine ENA-78, human chemokine eotaxin, human chemokine GRO alpha, human chemokine GROalpha, human chemokine GRObeta, human chemokine HCC-1, human chemokine HCC-1, human chemokine 1-309, human chemokine IP-10, human chemokine L105_{—}3, human chemokine L105_{—}7, human chemokine MIG, human chemokine MIG-beta protein, human chemokine MIP-1alpha, Human chemokine MIP1beta, Human chemokine MIP-3alpha, Human chemokine MIP-3beta, human chemokine PF4, human chemokine protein 331D5, human chemokine protein 61164, human chemokine receptor CXCR3, human chemokine SDF1alpha, human chemokine SDF1beta, human chemokine ZSIG-35, human Chr19Kine protein, human CKbeta-9, human CKbeta-9, human CX3C 111 amino acid chemokine, human DNAX interleukin-40, human DVic-1 C-C chemokine, human EDIRF I protein sequence, human EDIRF II protein sequence, human eosinocyte CC type chemokine eotaxin, human eosinophil-expressed chemokine (EEC), human fast twitch skeletal muscle troponin C, human fast twitch skeletal muscle troponin I, human fast twitch skeletal muscle Troponin subunit C, human fast twitch skeletal muscle Troponin subunit I Protein, Human fast twitch skeletal muscle Troponin subunit T, human fast twitch skeletal muscle troponin T, human foetal spleen expressed chemokine, FSEC, human GM-CSF receptor, human gro-alpha chemokine, human gro-beta chemokine, human gro-gamma chemokine, human IL-16 protein, human IL-1RD10 protein sequence, human IL-1RD9, human IL-5 receptor alpha chain, human IL-6 receptor, human IL-8 receptor protein hIL8RA, human IL-8 receptor protein hIL8RB, human IL-9 receptor protein, human IL-9 receptor protein variant #3, human IL-9 receptor protein variant fragment, Human IL-9 receptor protein variant fragment#3, human interleukin 1 delta, human Interleukin 10, human Interleukin 10, human interleukin 18, human interleukin 18 derivatives, human interleukin-1 beta precursor, human interleukin-1 beta precursor, human interleukin-1 receptor accessory protein, human interleukin-1 receptor antagonist beta, human interleukin-1 type-3 receptor, human Interleukin-10 (precursor), human Interleukin-10 (precursor), human interleukin-11 receptor, human interleukin-12 40 kD subunit, human interleukin-12 beta-1 receptor, human interleukin-12 beta-2 receptor, human Interleukin-12 p35 protein, human Interleukin-12 p40 protein, human interleukin-12 receptor, human interleukin-13 alpha receptor, human interleukin-13 beta receptor, human interleukin-15, human interleukin-15 receptor from clone P1, human interleukin-17 receptor, human interleukin-18 protein (IL-18), human interleukin-3, human interleukin-3 receptor, human interleukin-3 variant, human interleukin-4 receptor, human interleukin-5, human interleukin-6, Human interleukin-7, human interleukin-7, human interleukin-8 (IL-8), human intracellular IL-1 receptor antagonist, human IP-10 and HIV-1 gp120 hypervariable region fusion protein, human IP-10 and human Muc-1 core epitope (VNT) fusion protein, human liver and activation regulated chemokine (LARC), human Lkn-1 Full-Length and Mature protein, human mammary associated chemokine (MACK) protein Full-Length and Mature, human mature chemokine Ckbeta-7, human mature gro-alpha, human mature gro-gamma polypeptide used to treat sepsis, human MCP-3 and human Muc-1 core epitope (VNT) fusion protein, human MI10 protein, human MI1A protein, human monocyte chemoattractant factor hMCP-1, human monocyte chemoattractant factor hMCP-3, human monocyte chemotactic proprotein (MCPP) sequence, human neurotactin chemokine like domain, human non-ELR CXC chemokine H174, human non-ELR CXC chemokine IP10, human non-ELR CXC chemokine Mig, human PAI-1 mutants, human protein with IL-16 activity, human protein with IL-16 activity, human secondary lymphoid chemokine (SLC), human SISD protein, human STCP-1, human stromal cell-derived chemokine, SDF-1, Human T cell mixed lymphocyte reaction expressed chemokine (TMEC), human thymus and activation regulated cytokine (TARC), human thymus expressed, human TNF-alpha, human TNF-alpha, human TNF-beta (LT-alpha), human type CC chemokine eotaxin 3 protein sequence, human type II interleukin-1 receptor, human wild-type interleukin-4 (hIL-4) protein, human ZCHEMO-8 protein, humanized Anti-VEGF Antibodies, and fragments thereof, humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-11), IL-1 mature, IL-10 receptor, IL-11, IL-11, IL-12 p40 subunit, IL-13, IL-14, IL-15, IL-15 receptor, IL-17, IL-17 receptor, Il-17 receptor, Il-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, Il-4 mutein Y124G, IL-4 mutein Y124X, IL-4 muteins, Il-5 receptor, IL-6, Il-6 receptor, IL-7 receptor clone, Il-8 receptor, IL-9 mature protein variant (Met117 version), immunoglobulins or immunoglobulin-based molecules or fragment of either (e.g. a Small Modular ImmunoPharmaceutical™ (“SMIP”) or dAb, Fab′ fragments, F(ab′)₂, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon a species and sub-species, interferon β species and sub-species, interferon γ|species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL10, IL11 and IL2), interleukins (such as IL10, IL11 and IL2), Japanese encephalitis vaccine, Kalikrein Inhibitor, Keratinocyte growth factor, Kunitz domain protein (such as aprotinin, amyloid precursor protein and those described in WO 03/066824, with or without albumin fusions), Kunitz domain protein (such as aprotinin, amyloid precursor protein and those described in WO 03/066824, with or without albumin fusions), LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody not described herein, MP52, Mutant Interleukin 6 S176R, myofibrillar contractile protein Troponin I, Natriuretic Peptide, Nerve Growth Factor-beta, Nerve Growth Factor-beta2, Neuropilin-1, Neuropilin-2, Neurotactin, Neurotrophin-3, Neurotrophin-4, Neurotrophin-4-a, Neurotrophin-4-b, Neurotrophin-4-c, Neurotrophin-4-d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, plateletderived endothelial cell growth factor (PD-ECGF), Platelet-Derived Growth Factor A chain, Platelet-Derived Growth Factor B chain, polypeptide used to treat sepsis, Preproapolipoprotein “milano” variant, Preproapolipoprotein “pads” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, Retroviral protease inhibitors, Rotavirus Vaccine, RSV Mab, Secreted and Transmembrane polypeptides, Secreted and Transmembrane polypeptides, serum cholinesterase, serum protein (such as a blood clotting factor), Soluble BMP Receptor Kinase Protein-3, Soluble VEGF Receptor, Stem Cell Inhibitory Factor, Staphylococcus Vaccine, Stromal Derived Factor-1 alpha, Stromal Derived Factor-1 beta, Substance P (tachykinin), T1249 peptide, T20 peptide, T4 Endonuclease, TACI, Tarc, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor β, Troponin peptides, Truncated monocyte chemotactic protein 2 (6-76), Truncated monocyte chemotactic protein 2 (6-76), Truncated RANTES protein (3-68), tumour necrosis factor, Urate Oxidase, urokinase, Vasopressin (Neurophysin II), VEGF R-3; VEGF Receptor, KDR; fik-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9 and variants, fragments and analogues thereof.

The biological products may or may not include albumin fusions. Suitable albumin fusions include those described in U.S. Pat. No. 6,905,688 and include albumin fusions wherein the therapeutic protein fused to albumin is fused to biological product as described above.

As discussed above, the eleventh aspect of the present invention provides a method of culturfing mammalian cells, said method comprising incubating the cells in a cell culture media according to the ninth aspect of the invention. The cell culture media of the present invention may or may not be used for adherent cell culture, for suspension cell culture, or as a culture media for hybridoma cells, monoclonal antibody producing cells, virus-producing cells, transfected cells, cancer cells and/or recombinant peptide producing cells. The compositions may be used to culture eukaryotic cells, such as plant and/or animal cells. The cells may be mammalian cells, fish cells, insect cells, amphibian cells or avian cells. Other types of cells can be selected from the group consisting of MK2.7 cells (ATCC Catalogue No. CRL1909, an anti-murine-VCAM IgGl expressing hybridoma cell), HEK 293 cells, PER-C6 cells, CHO cells, COS cells, 5L8 hybridoma cells, Daudi cells, EL4 cells, HeLa cells, HL-60 cells, K562 cells, Jurkat cells, THP-1 cells, Sp2/0 cells; and/or the hybridoma cells listed in WO 2005/070120, table II hereby incorporated by reference or any other cell type disclosed herein or known to one skilled in the art.

Basal media may comprise, but are not limited to Dulbecco's Modified Eagle's Medium (DMEM), Minimal Essential Medium (MEM), Basal Medium Eagle (BME), RPMI 1640, F-10, F-12, a Minimal Essential Medium (αMEM), Glasgow's Minimal Essential Medium (G-MEM), and/or Iscove's Modified Dulbecco's Medium.

The present invention also provides a method of cultivating eukaryotic cells including contacting the cells with the compositions that are useful as cell culture medium of the present invention and/or maintaining the cells under conditions suitable to support cultivation of the cells in culture. In a particular embodiment, the cells are cancer cells or hybridoma cells. In other embodiments, methods of cultivating tissue explants are cultures are provided including contacting the tissues with the cell culture media compositions described herein.

In one embodiment, the method includes contacting hybridoma cells with a composition including: (i) basal media; (ii) recombinant albumin; (iii) glutamine; (iv) insulin (optionally recombinant insulin as discussed above); (v) recombinant protein comprising the sequence of a transferrin mutant of the invention; and/or (vi) ethanolamine, and/or maintaining the hybridoma cells under conditions suitable to support cultivation of the hybridoma cells in culture. In a specific embodiment, the method includes contacting hybridoma cells with a composition including (i) basal media; (ii) approximately 0.5% (w/v) albumin; (iii) approximately 4 mM glutamine; (iv) approximately 10 mg/L insulin; (v) approximately 1 mg/L recombinant protein comprising the sequence of a transferrin mutant of the invention; (vi) approximately 1 0 μM ethanolamine.

The present invention will now be exemplified with reference to the following non-limiting examples and figures.

### BRIEF DESCRIPTION OF THE FIGURES

FIGS. 1 to 10 show various plasmid maps for plasmids mentioned in the examples.

FIG. 11 shows RIE analysis of transferrin (S415A, T613A) secretion from various S. cerevisiae strains containing pDB2973 and pDB2974. 10 mL BMMD shake flasks were inoculated in triplicate with 3004 cryopreserved yeast stock and incubated for 4-days at 30° C. 54 culture supernatant loaded per well of a rocket immunoelectrophoresis gel. Plasma Tf standards concentrations are in μg/mL. 50 μL goat anti-Tf/50 mL agarose. Precipin was stained with Coomassie blue.

FIG. 12 shows SDS-PAGE analysis of recombinant transferrin (S415A, T613A) secreted from proprietary strains containing pDB2973 and pDB2974. 10 mL BMMD shake flasks were inoculated in triplicate with 300 μL cryopreserved yeast stock and incubated for 4-days at 30° C. 20 μL supernatant was analysed on non-reducing SDS-PAGE (4-12% NuPAGE®, MOPS buffer, InVitrogen) with GelCode® Blue reagent (Pierce).

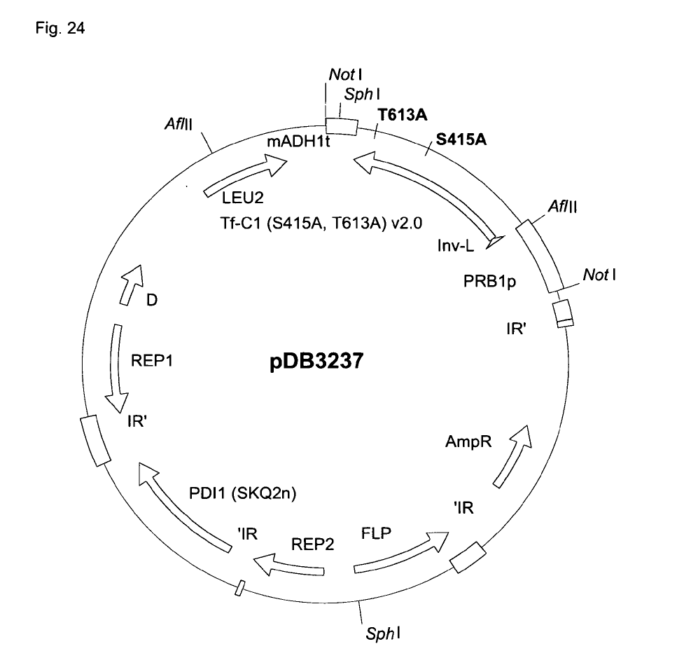

In Gel 1 of FIG. 12, the lanes correspond to the following samples: 1=20 μL SeeBlue Plus Markers; 2=20 μL Strain 1 pSAC35 s/n (negative control); 3=20 μL Strain 1 pDB2973 s/n; 4=20 μL Strain 1 ρDB2973 s/n; 5=20 μL Strain 2; 6=ρDB2973 s/n; 6=20 μL Strain 3 pDB2973 s/n; 7=20 μL Strain 4 pDB2973 s/n; 8=20 μL Strain 1 pDB2974 s/n; 8=20 μL Strain 1 pDB2929 s/n (positive control); 10=20 μL SeeBlue Plus Markers.

In Gel 2 of FIG. 12, the lanes correspond to the following samples: 1=20 μL SeeBlue Plus Markers; 2=20 μL Strain 1 pSAC35 s/n (negative control); 3=20 Strain 1 pDB2974 s/n; 4=20 μL Strain 1 pDB2974 s/n; 5=20 μL Strain 2 pDB2974 s/n; 6=20 μL Strain 3 pDB2974 s/n; 7=20 μL Strain 4 pDB2974 s/n; 8=20 μL Strain 1 pDB2973 s/n; 9=20 μL Strain 1 pDB2929 s/n (positive control); 10=20 μL SeeBlue Plus Markers.

FIG. 13 shows analytical TBE-urea gel analysis of recombinant transferrin (N413Q, N611Q) and transferrin (S415A, T613A). Samples were prepared according to the protocol described in the following example. 20 μg samples were separated on 6% TBE Urea PAGE (Invitrogen) and stained with Coomassie G250 (Pierce). Lanes 1-3 show Strain 1 [pDB2929] samples; Lanes 4-6 show Strain 1 [pDB2973] samples; Lanes 1 & 4 show purified recombinant transferrin mutants; Lanes 285 show recombinant apo-transferrin mutants; Lanes 386 show recombinant holo-transferrin mutants:

FIG. 14 shows the structure of plasmid pDB3191

FIG. 15 shows the structure of plasmid pDB3753

FIG. 16 shows the structure of plasmid pDB3768

FIG. 17 shows RIE analysis of recombinant transferrin (S415A, T613A), recombinant transferrin (S415C, T613A), recombinant transferrin (S415A, T613C), recombinant transferrin (S32A, S415A, T613A), and recombinant transferrin (S32C, S415A, T613A) secretion from S. cerevisiae strains Strain 1 containing pDB2973, pDB3773, pDB3765, pDB3768 or pDB3778 respectively. 10 mL BMMD shake flasks were inoculated in duplicate with 200 μL cryopreserved yeast stock and incubated for 5-days at 30° C. Duplicate samples of 4 μL culture supernatant were loaded per well of a rocket immunoelectrophoresis gel. Plasma Tf standards concentrations are in μg/mL. 30 μL goat anti-Tf/50 mL agarose. Precipin was stained with Coomassie blue.

Gel 1 of FIG. 17 shows RIE analysis of recombinant transferrin (S415A, T613A), recombinant transferrin (S415C, T613A), and recombinant transferrin (S415A, T613C) secretion from S. cerevisiae strains Strain 1 containing pDB3237, pDB3773 or pDB3765 respectively. Gel 2 of FIG. 17 shows RIE analysis of recombinant transferrin (S415A, T613A), recombinant transferrin (S32A, S415A, T613A), and recombinant transferrin (S32C S415A, T613A), secretion from S. cerevisiae strains Strain 1 containing pDB3237, pDB3768 or pDB3778 respectively.

FIG. 18 shows non-reducing SDS-PAGE analysis of recombinant transferrin (S415A, T613A), recombinant transferrin (S415C, T613A), recombinant transferrin (S415A, T613C), recombinant transferrin (S32A, S415A, T613A), recombinant transferrin (S32C, S415A, T613A) secretion from S. cerevisiae strains Strain 1 containing pDB2973, pDB3773, pDB3765, pDB3768 and pDB3778 respectively. 10 mL BMMD shake flasks were inoculated in duplicate with 2004 cryopreserved yeast stock and incubated for 5-days at 30° C. 20 μL supernatant was analysed on non-reducing SDS-PAGE (4-12% Bis/Tris NuPAGE®, MOPS buffer, Invitrogen) with GelCode® Blue reagent (Pierce).

In Gel 1 of FIG. 18, the lanes correspond to the following samples: 1=20 μL SeeBlue Plus Markers; 2=20 μL Strain 1 [pDB3237] s/n; 3=20 μL Strain 1 [pDB3237] s/n; 4=20 μL Strain 1 [pDB3773] s/n; 5=20 μL Strain 1 [pDB3773] s/n; 6=Strain 1 [pDB3765] s/n; 7=20 μL Strain 1 [pDB3765] s/n. Gel 2 of FIG. 18 the lanes correspond to the following samples: 1=20 μL SeeBlue Plus Markers; 2=no sample 3=20 μL Strain 1 [pDB3237] s/n; 4=20 μL Strain 1 [pDB3237] s/n; 5=20 μL Strain 1 [pDB3768] s/n; 6=20 μL Strain 1 [pDB3768] s/n; 7=Strain 1 [pDB3778] s/n; 8=20 μL Strain 1 [pDB3778] supernatant.

FIG. 19 shows analytical TBE-urea gel analysis of recombinant transferrin (S415A, T613A) and recombinant transferrin (S415C, T613A).

Samples were prepared according to the protocol described in the following example. 5 μg samples were separated on 6% TBE Urea PAGE (Invitrogen) and stained with Coomassie G250 (Pierce).

Lanes 1-2 shows Strain 1 [pDB3237] samples; Lane 3 shows Strain 1 [pDB3773] samples; Lane 1 shows iron-free recombinant transferrin (S415A, T613A) preparation; Lanes 2 and 3 shows iron-loaded recombinant transferrin mutants.

FIG. 20 shows analytical TBE-urea gel analysis of recombinant transferrin supernatants expressed from Strain 1 [pDB3237], Strain 1 [pDB3773] and Strain 1 [pDB3765]. compared to purified recombinant human transferrin (S415A, T613A) standard. Gel 1 in FIG. 20 Lane 1-2 shows purified recombinant transferrin (S415A, T613A) samples; Lanes 3-4 show Strain 1 [pDB3237] samples; Lanes 1 and 3 show iron-free preparations; Lanes 2 and 4 iron-loaded preparations. Gel 2 FIG. 20 Lanes 1-2 shows purified recombinant transferrin (S415A, T613A) samples; Lane 3-4 shows Strain 1 [pDB3773] samples; Lanes 1 and 3 show iron-free preparations; Lanes 2 and 4 iron-loaded preparations. Gel 3 in FIG. 20. Lanes 1-2 shows purified recombinant transferrin (S415A, T613A) samples; Lane 3-4 shows Strain 1 [pDB3765] samples; Lanes 1 and 3 show iron-free preparations; Lanes 2 and 4 shows iron-loaded preparations.

FIG. 21 shows analytical TBE-urea gel analysis of recombinant transferrin supernatants, expressed from Strain 1 [pDB3237], Strain 1 [pDB3778] and Strain 1 [pDB3768]. Gel 1 in FIG. 21 Lane 1-2 shows purified recombinant transferrin (S415A, T613A) samples; Lanes 3-4 show Strain 1 [pDB3768] samples; Lanes 1 and 3 show iron-free preparations; Lanes 2 and 4 shows iron-loaded preparations. Gel 2 FIG. 21 Lanes 1-2 shows Strain 1 [pDB3237] samples; Lane 3-4 shows Strain 1 [pDB3778] samples; Lanes 1 and 3 show iron-free preparations; Lanes 2 and 4 shows iron-loaded preparations.

FIG. 22 shows Surface Plasmon Resonance (SPR) analysis of purified iron-loaded preparations of recombinant transferrins (S415A, T613A) and recombinant transferrins (S415C, T613A).

FIG. 23 shows deconvolved mass spectra from analysis of recombinant transferrin (S415A, T613A), recombinant transferrin (S32C, S415A, T613A) and recombinant transferrin (S32A, S415A, T613A) using ESI-TOF mass spectrometry. Spectrum A shows the mass spectrum of recombinant transferrin (S415A, T613A) purified from high cell density fermentation of Strain 1 [pDB3237]. Peak identification A) unmodified molecule (theoretical mass 75098 Da), B) unmodified molecule+1 hexose (theoretical mass 75259 Da). Spectrum 8 shows the mass spectrum of recombinant transferrin (S32C, S415A, T613A) variant purified from high cell density fermentation of Strain 1 [pDB3778]. Peak identification C) unmodified molecule (theoretical mass 75114 Da). Spectrum C shows the mass spectrum of recombito nant transferrin (S32A, S415A, T613A) variant purified from high cell density fermentation of Strain 1 [pDB3768]. Peak identification D) unmodified molecule (theoretical mass 75130 Da).

FIG. 24 plasmid map of the plasmid pDB3237

### EXAMPLES

### Example 1

### Constructions of Expression Vectors

Expression plasmids were constructed for the production of unglycosylated recombinant transferrin having mutations to serine-415 and threonine-613 within the -N-X-S/T- motif. No significant differences were observed between the quantity or quality of the previouslydisclosed unglycosylated recombinant transferrin mutant N413Q, N611Q, when produced from a first S. cerevisiae strain (Strain 1) [pDB2929] and the new unglycosylated recombinant transferrin mutant S415A, T613A, when produced from Strain 1 [pDB2973], as determined by RIE, SDS-PAGE, urea gel analysis, mass spectrometry, N-terminal sequencing and iron delivery to human erythroleukemic cells grown in vitro.

Oligosaccharyl transferase catalyses the transfer of oligosaccharide chains from pyrophosphoryl dolichol to the asparagine residue within the sequence -Asn-X-Thr/Ser-, where X is any amino acid other than proline or aspartic acid (de Jong et al, 1990, Clin Chim Acta, 190, 1; Lau et al, 1983, J Biol Chem, 258, 15255). N-linked glycosylation of secreted proteins α-curs at this sequence motif within the endoplasmic reticulum.

However, due to steric constraints, only around one third of all possible sites within proteins are glycosylated. In human transferrin two possible sites are available, at asparagine-413 and asparagine-611 (both within the C-lobe), and both sites are utilised. Previous attempts to secrete human transferrin from S. cerevisiae lead to a diffuse heterogeneous product, believed to be due to hyper-mannosylation at asparagine-413 and asparagine-611 (data not shown), which is consistent with earlier observations relating the recombinant production of human transferrin in non-human host cells.

Production of a non-glycosylated recombinant transferrin mutant to prevent N-linked glycosylation at asparagine-413 and asparagine-611 by altering serine-415 and threonine-613 to alanine residues is described here.

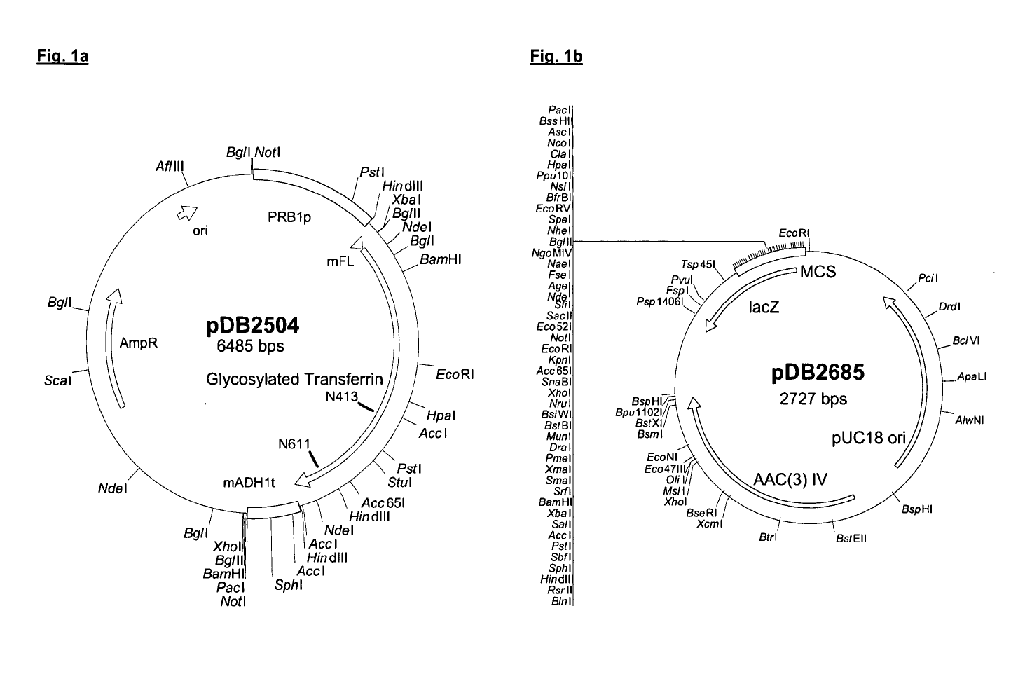

Plasmid pDB2504 (FIG. 1a) is pBST(+) (Sleep et al, 2001, Yeast, 18, 403-441) containing a NotI expression cassette for human transferrin, which is identical to the expression cassette in pDB2536 (FIG. 36 and Example 2 of WO 2005/061719, and Example 1 of WO 2005/061718) except that the codons for residues 413 and 611 of the mature transferrin protein are not mutated to prevent N-linked glycosylation, and are AAT and AAC respectively, encoding asparagine residues.

The codons for serine-415 and threonine-613 in the glycosylated human transferrin DNA sequence of pDB2504 were mutated to the preferred Saccharomyces cerevisiae codon for alanine, which was GCT (37%, http://www.yeastgenome.org/codon_usage.shtml). This was achieved according to the instruction manual of Stratagene's QuickChange™ Site-Directed Mutagenesis Kit. Mutagenic oligonucleotides CF156 (SEQ ID NO:4) and CF157 (SEQ ID NO: 5) were used to introduce the S415A mutation and mutagenic oligonucleotides CF158 (SEQ ID NO: 6) and CF159 (SEQ ID NO: 7) were used to introduce the T613A mutation (Table 1).

TABLE 1

| Name | Annotated Sequence |
|---|---|
| CF156 & | CF156 5′-GGCAGAAAACTACAATAAGGCTGATAATTGTGAGGATACACC-3′ |
| CF157 | CF157 3′-CCGTCTTTTGATGTTATTCCGACTATTAACACTCCTATGTGG-5′ |
| | A E N Y N K A D N C E D T |
| | <<< |
| | S415A |
| CF158 & | CF158 5′-GCACCTATTTGGAAGCAACGTAGCTGACTGCTCGGGCAACTTTTG-3′ |
| CF159 | CF159 3′-CGTGGATAAACCTTCGTTGCATCGACTGACGAGCCCGTTGAAAAC-5′ |
| | H L F G S N V A D C S G N F |
| | <<< |
| | T613A |

Mutagenesis was performed on a 1,154-bp HpaI-SphI pDB2504 fragment, which had been sub-cloned into the apramycin selectable E. coli cloning vector pDB2685 (FIG. 1b, also see WO 2005/061719), following digestion with HpaI, SphI and calf intestinal alkaline phosphatase. Competent E. coli DH5α were transformed with the ligation products and apramycin resistant colonies were selected (35 μg.mL⁻¹ apramycin). Plasmid pDB2958 (FIG. 2) was identified by restriction digestion with HpaI, SphI, EcoRI and NdeI.

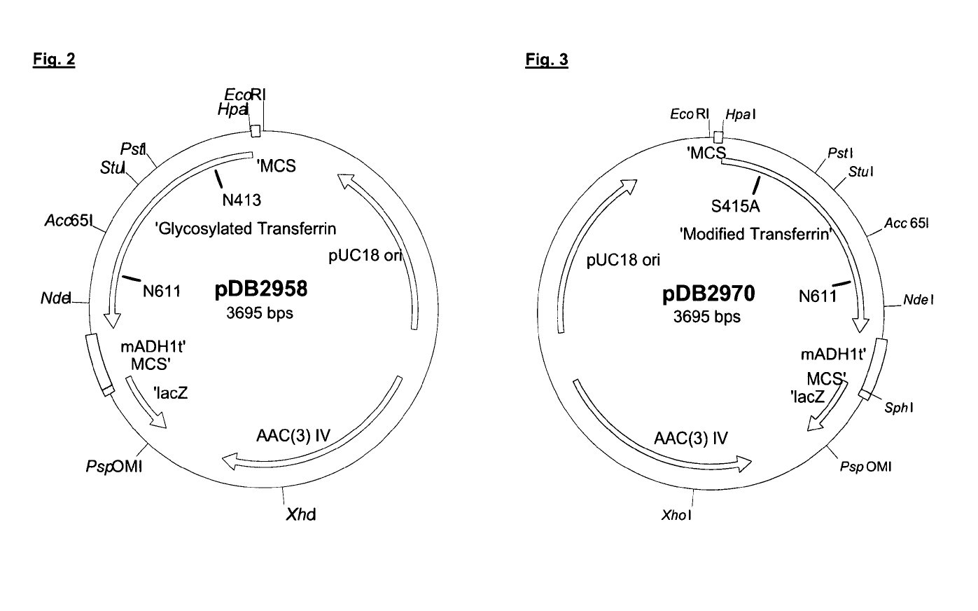

Plasmid pDB2958 was mutated with oligonucleotides CF156 and CF157 (Table 1) to introduce the S415A modification and produce plasmid pDB2970 (FIG. 3). Competent E. coli DH5α were transformed to apramycin resistance with the reaction products and plasmid DNA was isolated from four apramycin resistant colonies. These plasmids were subsequently mutated with oligonucleotides CF158 and CF159 to introduce the T613A modification and produce plasmid pDB2971 (FIG. 4). Apramycin colonies were isolated, and plasmid DNA was prepared from two clones originating from each of the four reactions used to introduce the S415A mutation. Three out of the eight plasmid preparations were selected initially for DNA sequencing to identify the S415A and T613A modification, each of which was derived from a separate T613A mutagenesis reaction.

DNA sequencing used oligonucleotides DS181 (SEQ ID NO: 8), DS182 (SEQ ID NO: 9), DS183 (SEQ ID NO: 10), DS184 (SEQ ID NO: 11), DS185 (SEQ ID NO: 12), DS186 (SEQ ID NO: 13), DS187 (SEQ ID NO: 14) M13 forward (SEQ ID NO: 15) and M13 reverse primers (SEQ ID NO: 16) (Table 2).

TABLE 2

| Primer | Description | Sequence |
|---|---|---|
| DS181 | Transferrin, | 5′-CTCAACCAGGCCCAGGA |
| | 24mer | ACATTTT-3′ |
| DS182 | Transferrin, | 5′-AGAGACCACCGAAG |
| | 19mer | ACTGC-3′ |
| DS183 | Transferrin, | 5′-AACCACTGCAGATTTGATG-3′ |
| | 19mer | |
| DS184 | Transferrin, | 5′-GCCAGAGCCCCGAATCAC-3′ |
| | 18mer | |
| DS185 | Transferrin, | 5′-ATTTTTCATATGTGTTTCTGTC-3′ |
| | 22mer | |
| DS186 | Transferrin, | 5′-TTCACAAAGGCCACATCTCC-3′ |
| | 20mer | |
| DS187 | Transferrin, | 5′-CAAAATACCCTGCCTCTG-3′ |
| | 18mer | |
| M13-F | l7mer | 5′-GTAAAACGACGGCCAGT-3′ |
| Ml3-R | 16mer | 5′-AACAGCTATGACCATG-3′ |

All three plasmids contained the expected S415A and T613A modifications, but one also contained an additional adenine insertion elsewhere within the 1,154-bp HpaI-SphI region. Consequently, the progenitor plasmid of one of the correct pDB2971 plasmid clones was sequenced with the same primers and shown to contain the expected pDB2970 sequence within the entire 1,154-bp HpaI-SphI region.

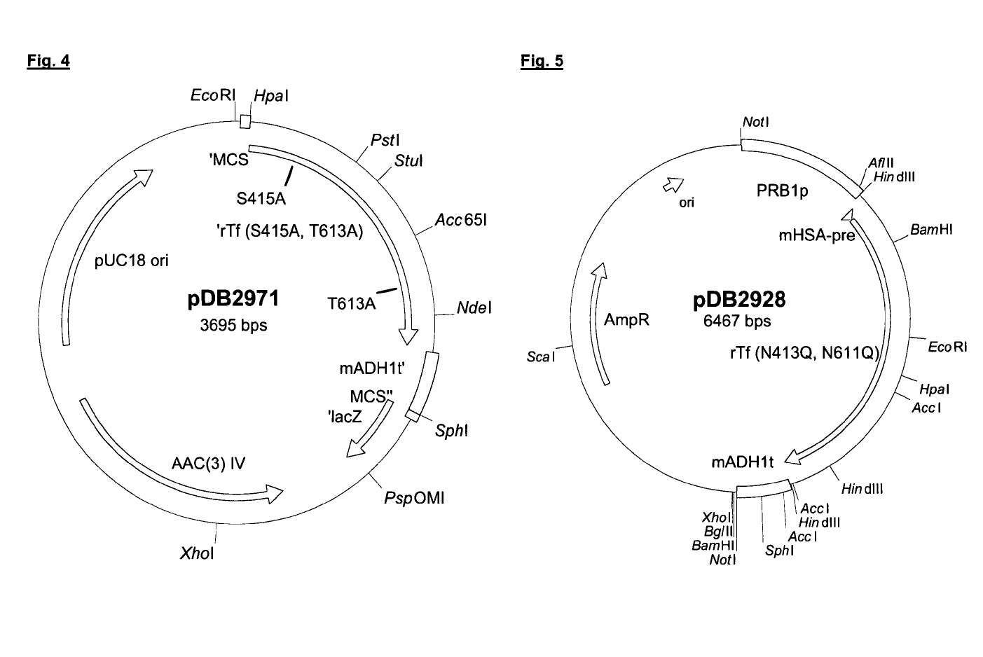

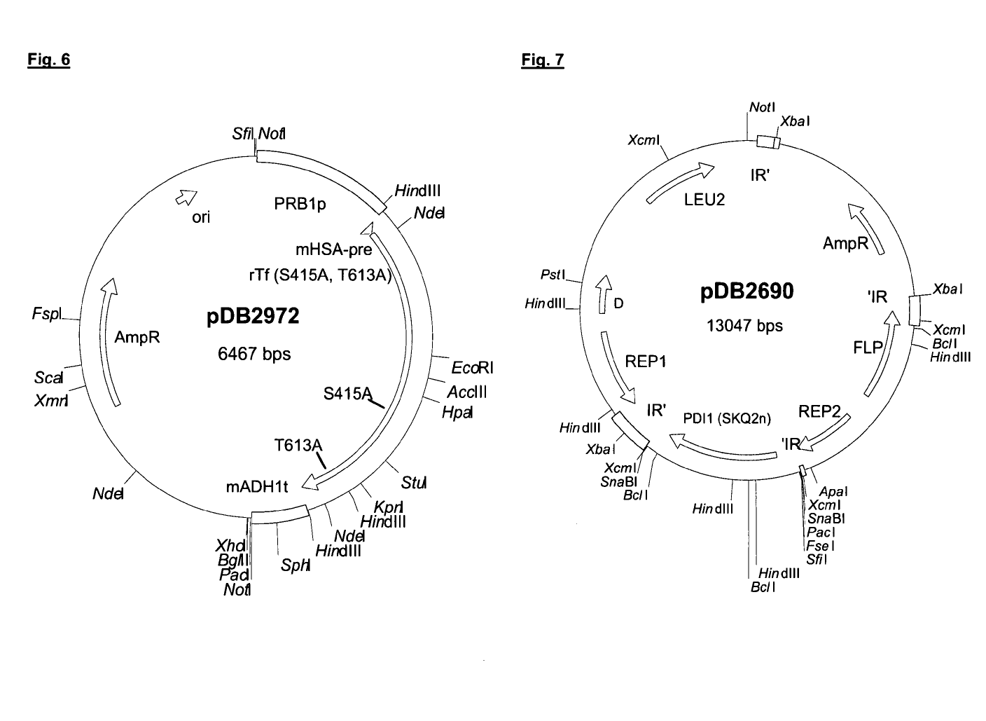

The 1,154-bp HpaI-SphI pDB2971 fragment containing the S415A and T613A modifications was isolated by gel purification and ligated with the 5,312-bp HpaI-SphI fragment from pDB2928 (FIG. 5, also see WO 2005/061718), which was purified following digestion with HpaI, SphI, AccI and calf intestinal alkaline phosphatase. The addition of AccI resulted in triple digestion of the unmodified 1,154-bp HpaI-SphI fragment. Competent E. coli DH5α were transformed to ampicillin resistance with the ligation products and plasmid DNA was prepared from selected clones. The pBST(+)-based plasmid, pDB2972 (FIG. 6), containing the NotI expression cassette for non-glycosylated recombinant human transferrin secretion using the mHSA-pre leader sequence was identified by restriction digestion with HpaI, SphI, NotI and NdeI.

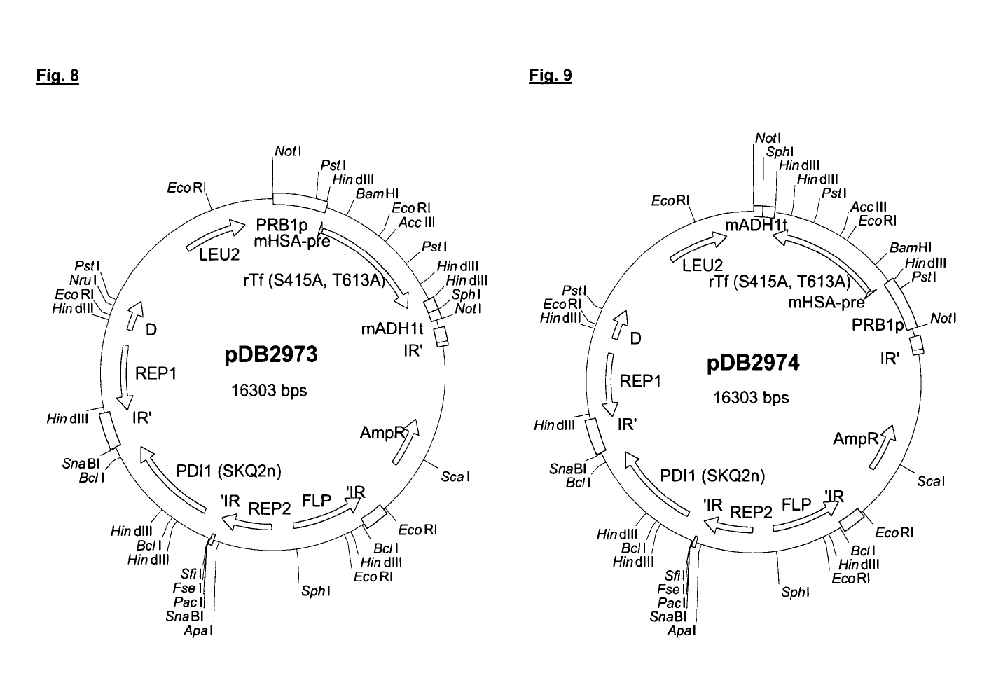

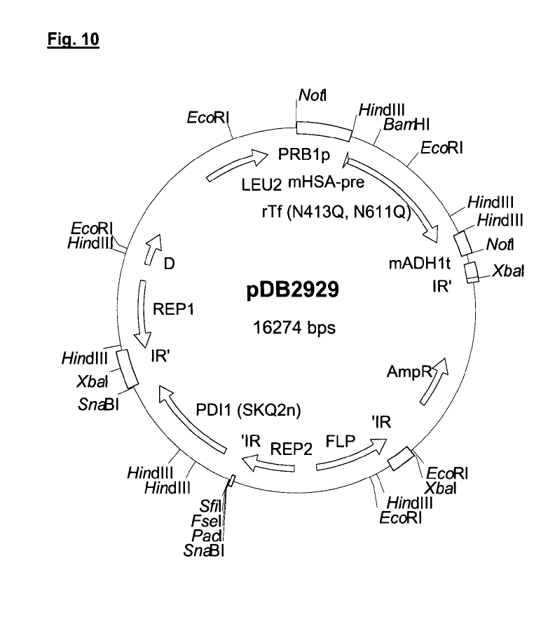

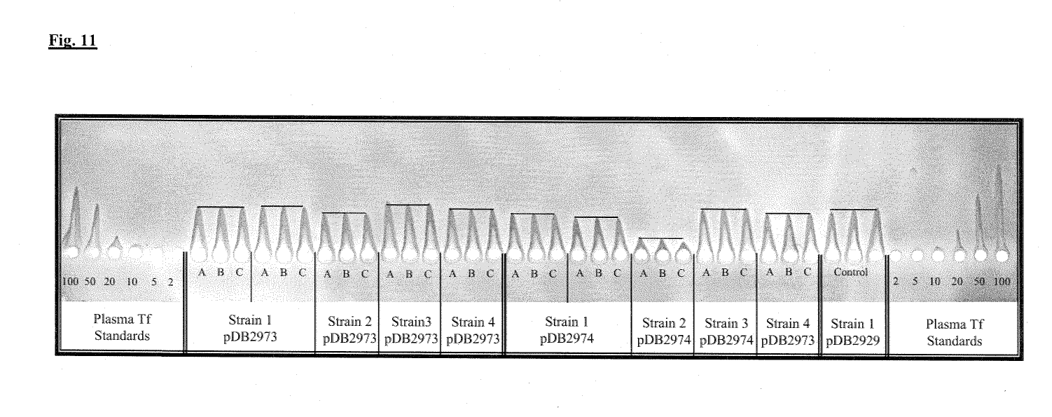

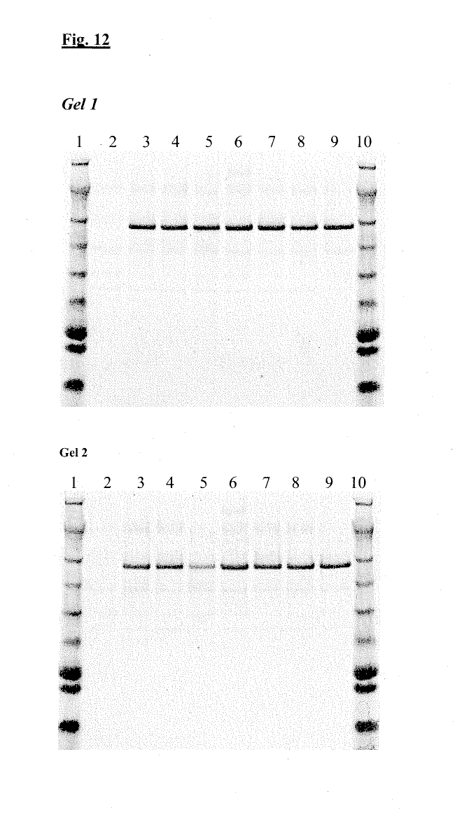

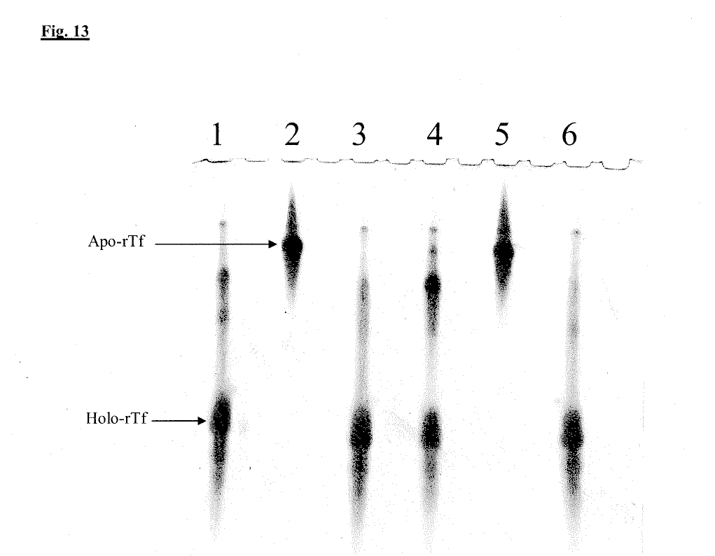

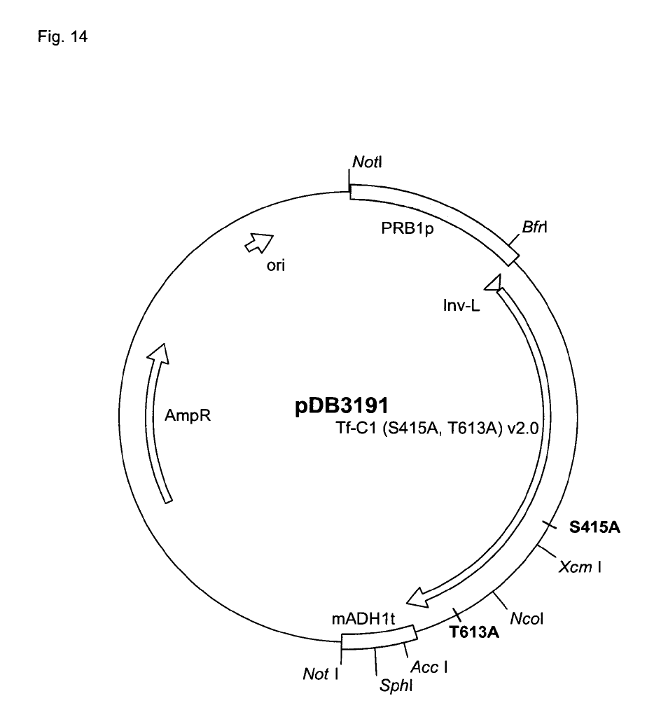

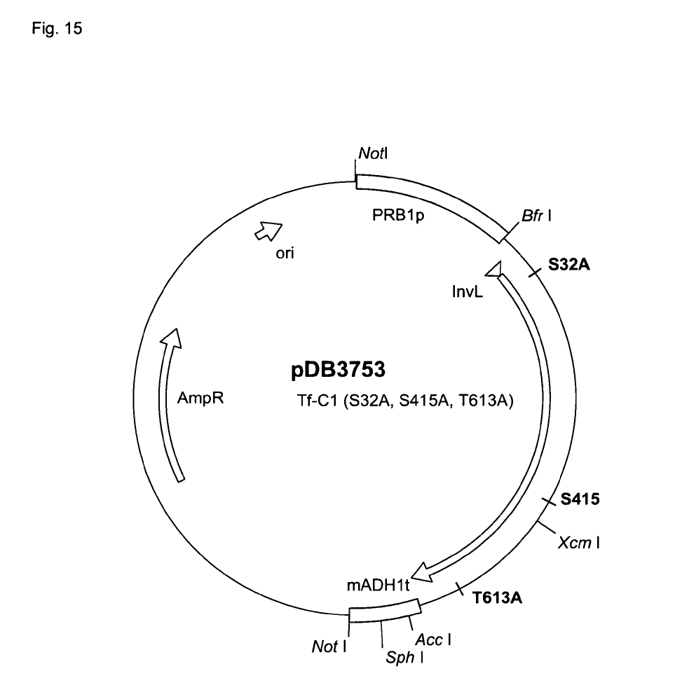

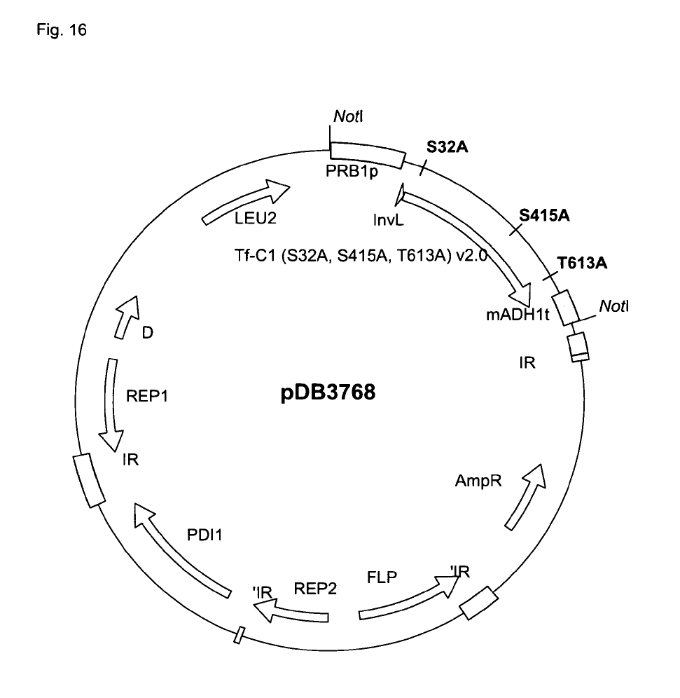

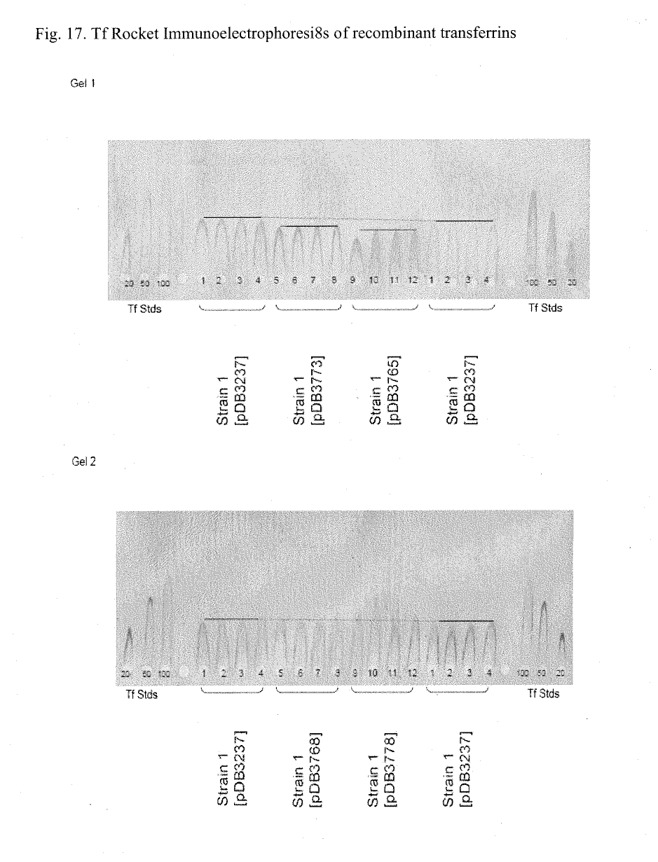

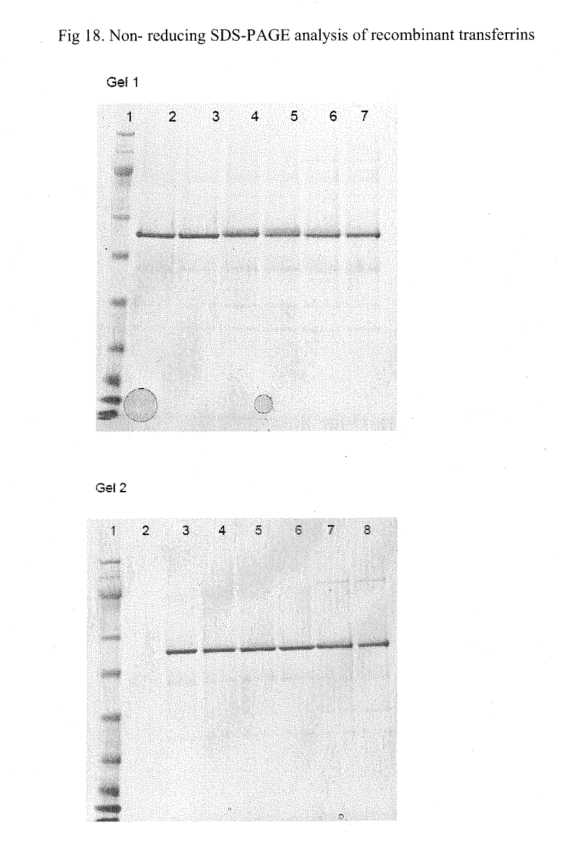

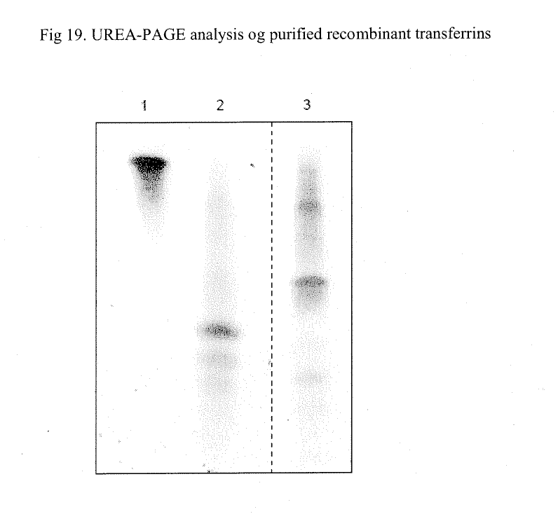

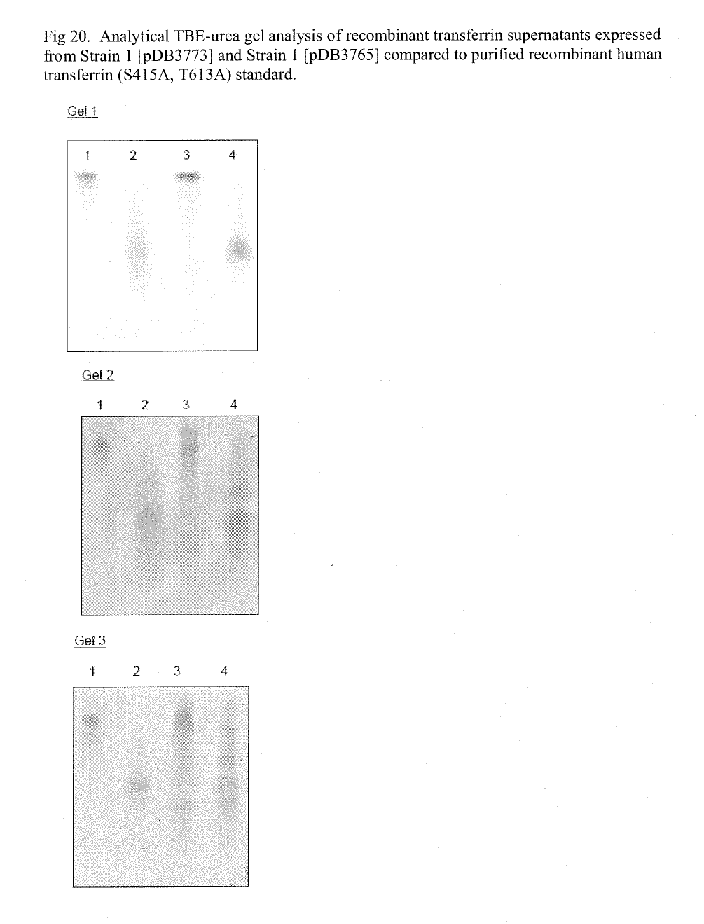

DNA sequencing with primers DS181, DS182, DS184, DS185, DS186 and DS187 (Table 2) confirmed the correct sequence of the 1,154-bp HpaI-SphI region and adjacent sequences. The 3,256-bp expression cassette was subsequently isolated from pDB2972 following digestion with NotI and ScaI. This was ligated with pDB2690 (FIG. 7, also see WO 2005/061718), which had been digested with NotI and calf intestinal alkaline phosphatase. Competent E. coli DH5α were transformed to ampicillin resistance with the ligation products and plasmid DNA was prepared from selected clones. Restriction digestion with HindIII, NotI, BamHI, NdeI and EcoRI was used to identify pDB2973 (FIG. 8) and pDB2974 (FIG. 9). The correct DNA sequence of the 1,154-bp HpaI-SphI region and adjacent sequences was confirmed for both plasmids. In pDB2973 the transferrin gene is transcribed in the same direction as LEU2, whereas in pDB2974 it is transcribed in the opposite direction.

### Example 2

### Production and Analysis of HST Mutants

A S. cerevisiae strain (Strain 1) was transformed to leucine prototrophy with pDB2973 and pDB2974. Yeast were transformed using a modified lithium acetate method (Sigma yeast transformation kit, YEAST-1, protocol 2; Ito et al, 1983, J. Bacteriol., 153, 16; Elble, 1992, Biotechniques, 13, 18). Transformants were selected on BMMD-agar plates, and subsequently patched out on BMMD-agar plates. The composition of BMMD is described by Sleep et al., 2002, Yeast, 18, 403. Cryopreserved stocks were prepared in 20% (w/v) trehalose from 10 mL BMMD shake flask cultures (24 hrs, 30° C., 200 rpm).

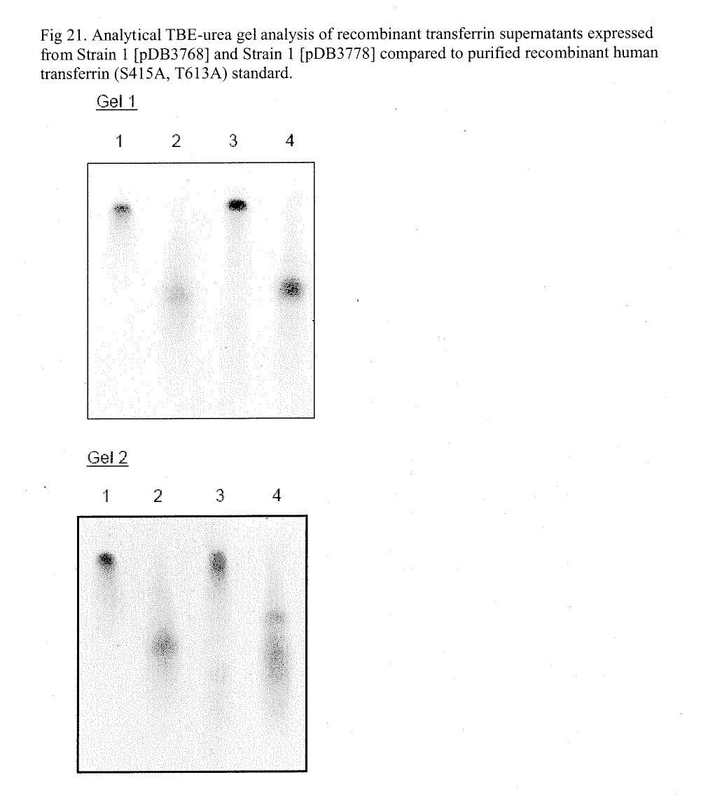

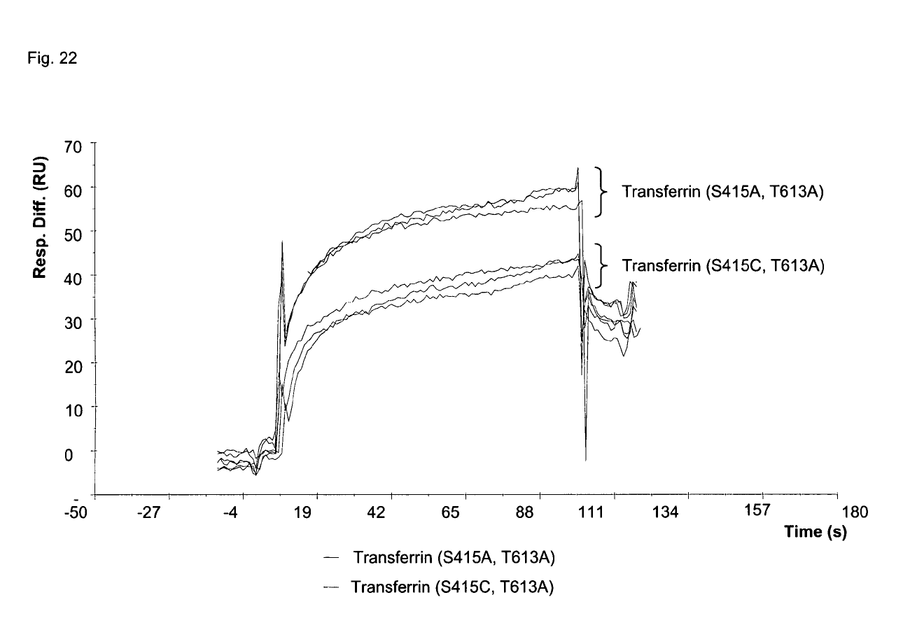

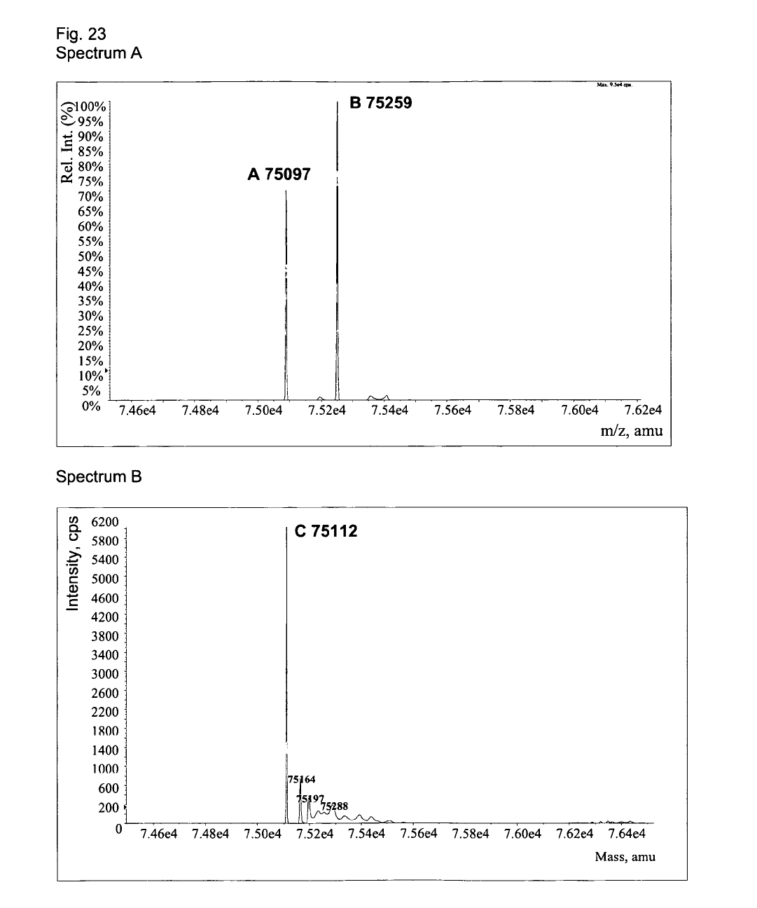

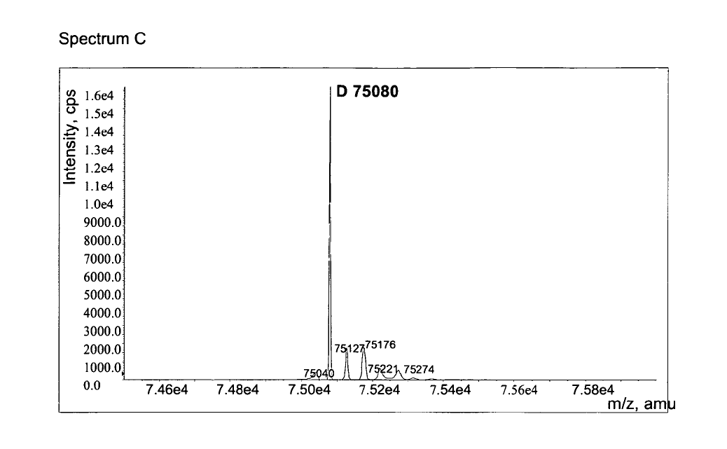

Triplicate 10 mL BMMD shake flask cultures were inoculated with each strain containing pDB2973 and pDB2974 and grown for 4-days at 30° C. Strain 1 [pDB2929] (FIG. 10, also see WO 2005/061718) was grown similarly for control purposes. pDB2929 contains the S. cerevisiae SKQ2n PCI1 gene and a N413Q, N611Q mutant transferrin gene that is transcribed in the same direction as LEU2. Supernatants were analysed by RIE and non-reducing SOS-PAGE. RIE analysis indicated that recombinant transferrin was secreted from all strains containing pDB2973 and pDB2974 (FIG. 11). The expression titres appeared to be marginally higher from all strains containing pDB2973 compared to pDB2974. The titres from pDB2973 and pDB2929 appeared to be equivalent.

Therefore, by RIE there appeared to be no significant difference between the levels of the alternative non-N-linked-glycosylated mutants secreted during shake flask culture of the strains studied.

Non-reducing SDS-PAGE analysis of recombinant transferrin (S415A, T613A) secretion is shown in FIG. 12. Various S. cerevisiae strains (Strains 1 to 4) containing pDB2973 and pDB2974 all secreted a proteinaceous band that co-migrated with the transferrin (N413Q, N611Q) band from Strain 1 [pDB2929], which was absent from the negative control strain. The yield of the transferrin (S415A, T613A) bands observed by SDS-PAGE agreed with the titres observed by RIE. Furthermore, by this SDS-PAGE analysis, there appeared to be no significant difference in the transferrin (S415A, T613A) band from Strain 1 [pDB2973] and the transferrin (N413Q, N611Q) band from Strain 1 [pDB2929]. No smearing of the transferrin (S415A, T613A) band was apparent, indicating that mutation of serine-415 and threonine-613 to alanine residues had successfully prevented hyperglycosylation at asparagine-413 and asparagine 611.

High cell density fermentation of Strain 1 [pDB2973] gave yields of −1.74 g.L⁻¹ (n=4), which was similar to the productivity of Strain 1 [pDB2929]. Characterisation of transferrin (S415A, T613A) from Strain 1 [pDB2973] indicated that it was functionally equivalent to transferrin (N413Q, N611Q) from Strain 1 [pDB2929]. During purification (SP-FF and DE-FF) and urea gel analysis (FIG. 13) the alternative non-glycosylated mutants appeared to be equivalent.

Urea gel electrophoresis was performed using a modification of the procedure of Makey and Seal (Monthony et al, 1978, Clin. Chem., 24, 1825-1827; Harris & Aisen, 1989, Physical biochemistry of the transferrins, VCH; Makey & Seal, 1976, Biochim. Biophys. Acta., 453, 250-256; Evans & Williams, 1980, Biochem. J., 189, 541-546) with commercial minigels (6% homogeneous TBE Urea, Invitrogen). Samples containing approximately 10 μg protein were diluted 1:1 in TBE-Urea sample buffer (Invitrogen), separated at 180 V for 550 to 600 Vh and stained with GelCode® Blue reagent (Pierce). Apo-transferrin was prepared by dialysis against 0.1 M citrate, 0.1 M acetate, 10 mM EDTA pH 4.5. Solutions were filtered (0.22 μm), concentrated to 10 mg/ml using a Vivaspin polyethersulphone 10,000 NMWCO centrifugal concentrator and diafiltered against 10 volumes water followed by 10 volumes of 0.1 M HEPES, 0.1 M NaHCO₃ pH 8.0. Samples were recovered from the concentrator with a rinse and made up to a final concentration of 5 mg/ml. Reconstituted holo-transferrin was prepared from this solution by addition of 10 μl 1 mM FeNTA (prepared freshly as an equimolar solution of ferric chloride in disodium nitrilotriacetic acid) to a 50 μl aliquot and allowed to stand for 10 minutes to permit CO₂ dissolution for completion of iron binding before electrophoretic analysis. This technique separates four molecular forms with different iron loadings namely (in order of increasing mobility) apo-transferrin, C-lobe and N-lobe bound monoferric transferrins and holo-transferrin. Separation of the four forms of transferrin is believed to be due to partial denaturation in 6M urea; where iron binding in any lobe causes a change in conformation resulting in increased resistance to denaturation. Thus the presence of iron in a lobe results in a more compact structure with higher electrophoretic mobility. Since the N-lobe has fewer disulphide bonds than the C-lobe (8 versus 11 respectively) it unfolds further in the absence of iron, making the monoferric form with iron bound to the C-lobe the least mobile.

Mass spectrometry identified the expected mass difference between the different non-glycosylated transferrin mutants and provided good evidence for the correct primary protein sequence in transferrin (S415A, T613A) (data not shown). Transferrin (S415A, T613A) was also comparable to transferrin (N413Q, N611Q) with respect to post-translational modification (data not shown).

Furthermore, recombinant transferrin (S415A, T613A) from Strain 1 [pDB2973] was equivalent to transferrin (N413Q, N611Q) from Strain 1 [pDB2929] its the ability to deliver iron to K562 cells in vitro (Table 3).

TABLE 3
Total iron uptake, unspecific uptake, apparent affinity and correlation
coefficient (r²) from human plasma control and recombinant transferrins
by human erythroleukemic K562 cells grown in vitro

| | Maximal | Unspecific | Apparent | |
|---|---|---|---|---|
| Sample | Uptake | Uptake. | Affinity | r² |
| Human Plasma | 2093 ± 83 | 313 ± 71 | 186 | 0.9976 |
| Transferrin | | | | |
| Strain 1 [pDB2929] | 1856 ± 106 | 355 ± 79 | 123 | 0.9946 |
| Strain 1 [pDB2973] | 1681 ± 117 | 362 ± 87 | 123 | 0.9923 |

Uptake data in fmol Fe/million cells 25 min, apparent affinity in nM transferrin (estimated concentrations not adjusted for systematic error)

It is to be noted that, although the maximal uptake appears higher for the control, this figure is not relevant. The maximal uptake is always that of the native transferrin control, thus the difference to the recombinant samples is a statistical deviation. The only important figures are the apparent affinity constants, which are all slightly lower than that of native transferrin, and the correlation coefficient representing the quality of the experimental data. In short, one could say that all these recombinant transferrins are at least as good as the native one in their ability to deliver iron to erythroid cells.

The data in Table 3 are obtained from a competition assay, where plasma transferrin was radiolabelled with iron-55, and the two unlabelled recombinant transferrin mutants were compared in their ability to inhibit iron-55 delivery by the radiolabelled iron-55.

K562 erythroleukemic cells, cultured in RPMI cell culture medium under standard conditions

(bicarbonate-buffered, 5% CO₂, antibiotics, 10% fetal calf serum) were washed with serum-free medium containing HEPES-buffer and 1 mg/ml of bovine serum albumin and used at a concentration of 10 million cells/ml in this medium.

Increasing concentrations of native or the respective diferric recombinant transferrin sample (0, 25, 100, 200, 400, 800, 1600 nM) were mixed with 100 nM of native diferric plasma transferrin labeled with ⁵⁵Fe in 25 μl of medium. Unlabeled native diferric transferrin served as control.

The reaction was started by the addition of 300 μl of cell suspension. After 25 min at 37° C. the reaction was stopped by immersion into an ice-bath, three aliquots of 60 μl of cell suspension were transferred to new tubes and the cells were centrifuged in the cold and again after addition of an oil layer of diethylphtalate/dibutylphthalate. The supernatant was removed, the cell pellet transferred into a counter vial and lysed with 0.5 M KOH+1% Triton X-100. The lysates were neutralized with 1M HCl after o/n lysis, mixed with Readysolv scintillation cocktail and counted in the Packard Liquid Scintillation Counter.

Therefore, mutation of serine-415 and threonine-613 appeared to be a viable alternative to mutation of asparagine residues in the -N-X-S/T- motif for the prevention of N-linked glycosylation of recombinant transferrin secreted from S. cerevisiae.

Previous studies have concluded that the N413Q, N611Q, transferrin mutant has biological equivalence to non-mutated transferrin (data not shown), and these studies show that mutation of serine-415 and threonine-613 results in a transferrin mutant with biological equivalence to the N413Q, N611Q transferrin mutant. It can, therefore, be concluded that mutation to of serine-415 and threonine-613 results in a transferrin mutant with biological equivalence to non-mutated transferrin.

### Example 3

### Construction of Transferrin Mutein Expression Plasmids

### A: Construction of Transferrin Mutein Expression Plasmids

Expression plasmids for transferrin variants of this invention can be constructed in similarity with the following description for Tf variant S415A, T613A.

Transferrin muteins are made by modification of a plasmid called pDB3237 by site directed mutagenesis. Overlapping mutagenic oligonucleotide sequences will be used to modify the codon of the selected residue(s) to any DNA sequence which encodes a cysteine residue (TGT or TGC) using the procedures indicated by a commercially available kit (such as Stratagene's Quikchanger™ Kit).

B: Construction of Transferrin (S415A. T613A) Expression Plasmid, pDB3237

Overlapping oligonucleotide primers are used to create a synthetic DNA encoding the invertase leader sequence and human transferrin (S415A, T613A) which is codon optimised for expression in S. cerevisiae.

SEQ ID NO: 18 comprises the mature human transferrin C_{I} variant protein encoding sequence modified at serine 415 and threonine 613 to alanine residues to prevent N-linked glycosylation at the Asn413 and Asn611 sites (nucleotides 124-2160); two translation stop codons (nucleotides 2161-2166); the invertase leader (signal) protein encoding sequence (nucleotides 67-123); the 3′ UTR and part of the ADH1 gene terminator up to an SphI cloning sites (nucleotides 2167-2359); the 5′ UTR and part of the PRB1 gene promoter up to an AfiII cloning sites (nucleotides 1-66).

The invertase leader (signal) protein encoding sequence (nucleotides 67-123) encodes the signal peptide MLLQAFLFLLAGFAAKISA (SEQ ID NO: 19)

The invertase leader sequence human transferrin (S415A, T613A) DNA sequence, is digested to completion with SphI and AflII to create a 2.357 kb fragment. Plasmid pDB2241 (4.383 kb), described in WO 00/44772 is digested to completion using restriction endonucleases SphI and AflII to create a 4.113 kb fragment, which is subsequently dephosphorylated using calf alkaline intestinal phosphatase. The 2.537 kb invertase leader sequence human transferrin (S415A, T613A) DNA fragment is ligated into the 4.113 kb SphI/AflII fragment from pDB2241 to create plasmid pDB3191 (FIG. 14). Plasmid pDB3191 is digested to completion with Nod restriction endonuclease to release the 3.259 kb invertase leader sequence human transferrin (S415A, T613A) expression cassette.

The construction of plasmid pDB2690 is described in WO/2005061719 A1. Plasmid pDB2690 (13.018 kb) is digested to completion with restriction endonuclease NotI and dephosphorylated using calf alkaline intestinal phosphatase and ligated with the 3.259 kb NotI transferrin (S415A, T613A) expression cassette to produce 16.306 kb pDB3237 which has the transferrin (S415A, T613A) expression cassette in the opposite direction to the LEU2 gene (FIG. 24).

Alternatively expression plasmids for transferrin variants of this invention can be made by subcloning synthesised DNA fragments into plasmid pDB3191 (FIG. 14) prior to subcloning of NotI transferrin variant expression cassettes into pDB2690

The transferrin DNA sequence of pDB3191 (FIG. 14) contains unique AflII, XcmI, NcoI and AccI restriction endonuclease sites. The positions of the proposed mutations were mapped on the transferrin expression cassette sequence of pDB3191 (FIG. 14). The sequence is flanked by AflII and XcmI restriction endonuclease sites to facilitate cloning. Further thiotransferrin variants were created by modification of the DNA sequence between the AflII and XcmI restriction site containing part of the mature human transferrin C₁ variant protein encoding sequence modified at serine 415 to alanine to prevent N-linked glycosylation at the Asn413 site up to an XcmI cloning site (nucleotides 124-1487); the invertase leader (signal) protein encoding sequence (nucleotides 67-123) and part of the PRB1 gene promoter up to an AflII cloning site (nucleotides 1-66). In the examples given codon TGT was used for the cysteine residue, however, codon TGC could also be used in this invention.

SEQ ID NO; 18, comprises part of the mature human transferrin C₁ variant protein encoding sequence modified at serine-32 to alanine to prevent O-linked glycosylation (nucleotides 216-218), and modified at serine-415 to alanine to prevent N-linked glycosylation at the Asn413 site up to an XcmI cloning site (nucleotides 124-1487); the invertase leader (signal) protein encoding sequence (nucleotides 67-123) and part of the PRB1 gene promoter up to an AflII cloning site (nucleotides 1-66). In this example codon optimized DNA was used, however, non-codon optimized DNA could also be used in this invention.

The SEQ ID NO: 18 variant DNA sequence was digested to completion with AflII and XcmI to create a 1.479 kb fragment. Plasmid pDB3191 (6.47 kb) was digested to completion using restriction endonucleases AflII and XcmI to create a 4.991 kb fragment, which was subsequently dephosphorylated using shrimp alkaline phosphatase. The 1.479 kb transferrin (S32A, S415A) variant DNA fragment was sublconed into the 4.991 kb AfflII/XcmI fragment from pDB3191 to create plasmids pDB3753 (FIG. 15).

The Transferrin (S32A, S415A, T613A) variant subcloning plasmid pDB3753 was digested to completion with NotI restriction endonuclease to release the appropriate 3.259 kb Transferrin (S32A, S415A, T613A) expression cassette.

The construction of plasmid pDB2690 has been described in WO/2005061719 A1. Plasmid pDB2690 (13.018 kb) was digested to completion with restriction endonuclease NotI and dephosphorylated using shrimp alkaline phosphatase and ligated with the 3.259 kb NotI Transferrin (S32A, S415A, T613A) variant expression cassette to produce 16.306 kb plasmids pDB3768 which has the Transferrin (S32A, S415A, T613A) variant expression cassette in the same orientation to the LEU2 gene (FIG. 15).

A S. cerevisiae strain (Strain 1) was transformed to leucine prototrophy with plasmids pDB3237 or pDB3768. Yeast were transformed using a modified lithium acetate method (Sigma yeast transformation kit, YEAST-1, protocol 2; Ito et al, 1983, J. Bacteriol., 153, 16; Elble, 1992, Biotechniques, 13, 18). Transformants were selected on BMMD-agar plates, and subsequently patched out on BMMD-agar plates. The composition of BMMD is described by Sleep et al., 2002, Yeast, 18, 403. Cryopreserved stocks were prepared in 20% (w/v) trehalose from 10 mL BMMD shake flask cultures (24 hrs, 30° C., 200 rpm).

C: Construction of Transferrin (S415C, T613A) Expression Plasmid, pDB3773

This plasmid was constructed using a method corresponding to the method for constructing pDB3237

D: Construction of Transferrin (S415A. T613C) Expression Plasmid pDB3765

This plasmid was constructed using a method corresponding to the method for constructing pDB3237

E: Construction of Transferrin (S32C, S415A, T613A) Expression Plasmid pDB3778

This plasmid was constructed using a method corresponding to the method for constructing pDB3237

### Example 4

### Productivity of Yeast Strain Expressing Recombinant Transferrin Mutants

Duplicate 10 mL BMMD shake flask cultures were inoculated with Strain 1 yeast strain containing pDB3237, pDB3773 pDB3765, pDB3778 and pDB3768 and grown for 5-days at 30° C. Supernatants were analysed by rocket immuno-electrophoresis (RIE) and non-reducing SOS-PAGE. RIE analysis indicated that recombinant transferrin was secreted from all strains containing pDB3237, pDB3773, pDB3765, pDB3768 and pDB3778 (FIG. 17 and FIG. 18). The expression titres appeared similar from Strain 1 containing pDB3237 expressing recombinant transferrin mutant S415A, T613A, when compared to Strain 1 containing pDB3778 and pDB3768 expressing recombinant transferrin mutant S32C S415A, T613A and recombinant transferrin mutant S32A, S415A, T613A respectively (Gel 2 in FIG. 17), indicating that mutation of serine-32 in these constructs does not reduce the product yield. In contrast, expression titres appeared lower from Strain 1 containing pDB3773 or pDB3765 expressing recombinant transferrin mutant S415C, T613A or recombinant transferrin mutant S415A, T613C respectively (Gel 1 in FIG. 17), indicating that the mutations of serine and threonine to alanine residues for the prevention of N-linked glycosylation is preferred.

Therefore, by RIE there appeared to be no significant difference between the levels of recombinant transferrin mutants containing the S415A and T613A mutation and an additional mutation at serine-32 when compared to recombinant transferrin mutant containing only the S415A and T613A mutation. Similarly, by SDS-PAGE analysis (Gel 2 in FIG. 18), there appeared to be no significant difference in the recombinant transferrin (S415A, T613A) band from Strain 1 [pDB3237], compared to the recombinant transferrin (S32C, S415A, T613A) band from Strain 1 [pDB3778], or the recombinant transferrin (S32A, S415A, T613A) band from Strain 1 [pDB3768].

However, by RIE analysis, when a recombinant transferrin mutant was expressed containing a ‘non-conservative’ mutation such as a serine-415 substituted for cysteine or threonine-613 substitution for cysteine a reduced amount of recombinant protein was secreted. This was confirmed by SOS-PAGE analysis (Gel 1 in FIG. 18).

High cell density fed-batch fermentation of Strain 1 [pDB3768] expressing transferrin (S32A, S415A, T613A) variant gave a yield of 2.26 mg.mL⁻¹ and Strain 1 [pDB3768] expressing transferrin (S32C, S415A, T613A) gave a yield of 1.95 mg.mL⁻¹ which are similar to that seen for Strain 1 [pDB3237] expressing transferrin (S415A, T613A) variant. However, high cell density fed-batch fermentations of Strain 1 [pDB3773] expressing transferrin (S415C, T613A) gave a yield of ˜1.06 mg.mL⁻¹ (n=2) indicating that a “non-conservative” substitution of serine-415 to a cysteine residue results in significantly reduced productivity.

### Example 5

### Iron Binding Capability of Recombinant Transferrin (S415A, T613A) Compared to Recombinant Transferrin (S415C, T613A) and Recombinant Transferrin (S415A, T613C)

The iron binding capability of the recombinant transferrin (S415A, T613A) compared to recombinant transferrin (S415C, T613A) and recombinant transferrin (S415A, T613C) purified from shake flask supernatant of Strain 1 [pDB3237], Strain 1 [pDB3773] and Strain 1 [pDB3765] was compared to that of purified recombinant human transferrin (S415A, T613A) standard.

To iron-load purified recombinant transferrin (S415A, T613A) and recombinant transferrin (S415C, T613A) the following method was used. Sodium bicarbonate was added to purified transferrin to give a final concentration of 20 mM. The amount of iron (in the form of ammonium iron citrate at 10 mg.mL⁻¹ (16.5-18.5% Fe) to target 2 mol Fe³⁺.mol⁻¹ transferrin was calculated, added to the recombinant transferrin/20 mM Sodium bicarbonate preparation and allowed to mix for a minimum of 60 minutes at ambient temperature followed by ultrafiltration into 145 mM NaCl.

To prepare iron-free purified recombinant transferrin (S415A, T613A) the sample was incubated in 0.1M sodium citrate, 0.1 M sodium acetate, 10 mM EDTA pH 4.5 for a minimum of 180 minutes at ambient temperature, followed by ultrafiltration into 100 mM HEPES, 10 mM sodium carbonate buffer pH 8.0.

5 μg samples were separated on 6% TBE Urea PAGE (Invitrogen) and stained with Coomassie G250 (Pierce) (FIG. 19). The iron binding capability of purified recombinant transferrin (S415C, T613A) appeared different to recombinant transferrin (S415A, T613A) under these experimental conditions. Recombinant transferrin (S415C, T613A) sample (lane 3 in FIG. 19) which had been subjected to the same holisation treatment did not appear to be fully saturated with iron and showed bands that migrated through the analytical TBE Urea gel more slowly than recombinant transferrin (S415A, T613A) (lane 2 in FIG. 19) indicating that the recombinant ‘holo-transferrin’ (S415C, T613A) sample did not contain the same amount of iron as the recombinant ‘holo-transferrin’ (S415A, T613A).

Recombinant transferrin variants expressed as shake flask supernatant after 5 days growth in 200 mL BMMD shake flask were isolated from yeast biomass by centrifugation. The supernatant samples were concentrated to 1 mL and diafiltered into 10 mM HEPES buffer pH 8. The material was assayed by RP-HPLC to obtain protein concentration and was subsequently split into two samples. One sample was converted to the apo-transferrin form by a two fold dilution and incubation in 0.1M sodium citrate, 0.1 M sodium acetate, 10 mM EDTA pH 4.5 for three hours, the other sample was treated by a procedure capable of converting apo-transferrin to the diferric holo-transferrin form (iron bound) by a two fold dilution in holoising buffer (0.5 M carbonate, 2.5 mg.mL⁻¹ iron citrate) for three hours.

0.5 μg samples were separated on 6% TBE Urea PAGE (Invitrogen) and stained with Coomassie G250 (Pierce). The iron binding capability of recombinant transferrin (S415A, T613A) (lane 3 and 4 in Gel 1 of FIG. 19) isolated from shake flask supernatant appeared to have the same iron loaded recombinant transferrin (S415A, T613A) (lane1 and 2 in Gel 1 of FIG. 20).

The iron binding capability of recombinant transferrin (S415C, T613A) and recombinant transferrin (S415A, T613C) isolated from shake flask supernatant of Strain 1 [pDB3773] and Strain 1 [pDB3765] respectively appeared different to that of recombinant transferrin (S415A, T613A) isolated from shake flask supernatant of Strain 1 [pDB3237] under these experimental conditions. Recombinant transferrin (S415C, T613A) samples (lane 4 in Gel 2 FIG. 20) which had been subjected to holoisation treatment to load transferrin with iron migrated through the analytical TBE urea gel with some species having reduced mobility compared to purified recombinant transferrin (S415A, T613A) (lane 2 in Gel 2 of FIG. 20) indicating that recombinant ‘holo-transferrin’ (S415C, T613A) was partially saturated with iron and not homogeneous with some of the recombinant transferrin (S415C, T613A) having 2 moles of iron bound and some the recombinant transferrin (S415C, T613A) having less 2 mole of iron bound.

Recombinant transferrin (S415A, T613C) samples (lane 4 in Gel 3 FIG. 20) which had been subjected to holoisation treatment to load transferrin with iron also migrated through the analytical TBE urea gel with some species having reduced mobility compared to purified recombinant transferrin (S415A, T613A) (lane 2 in Gel 3 of FIG. 20) indicating that recombinant ‘holo-transferrin’ (S415A, T613C) was not homogeneous in this analysis and that some the recombinant transferrin (S415A, T613C) had 2 moles of iron bound and some the recombinant transferrin (S415A, T613C) less than 2 mole of iron bound.

By analytical TBE urea gel electrophoresis there are appear to be functional differences in the iron binding capability of recombinant transferrin (S415A, T613A) compared to recombinant transferrin (S415C, T613A) and recombinant transferrin (S415A, T613C). This indicated that the recombinant transferrin (S415A, T613A) mutant was preferred for the control of N-linked glycosylation.

### Example 6

### Receptor Binding Capability of Recombinant Transferrin (S415A, T613A) Compared to Recombinant Transferrin (S415C, T613A)

The receptor binding capability of the recombinant transferrin variants was assessed by Surface Plasmon Resonance (SPR) analysis. The binding activity of a transferrin sample to transferrin receptor can be measured using surface plasmon resonance (SPR) a non-invasive optical technique in which the SPR response is a measure of change in mass concentration at the detector surface as molecules bind or dissociate. A sample is sent onto the surface of the sensor chip via a micro flow system at a constant flow rate. In this analysis, if the transferrin sample is able to bind to the TfR the mass on the surface sensor chip is increased due to binding between TfR and Tf molecules creating a surface plasmon wave and a shift of the SPR signal proportional to the binding quantity can be detected as a change in the resonance unit (RU). A response of 1 RU is equivalent to change in a surface concentration of about 1 pg.mm⁻².

Biacore sensor chips for interaction analysis between transferrin and the transferrin receptor were prepared by first immobilizing Transferrin receptor antibody prior to addition of the transferrin. Specifically, anti-Transferrin receptor (anti-TfR) antibody was immobilized to the CM5 sensor chip surface (GE Healthcare catalogue number BR-1000-14) using amine coupling chemistry at 25° C. The carboxymethylated dextran surface on a CM5 sensor chip flow cell were converted to active succinamide esters by the addition of N-hydroxysuccinimide:N-ethyl-N′-(dimethylaminopropyl) carbodiimide (NHS:EDC).

The (Transferrin) receptor specific binding can be confirmed by concurrently preparing a sensor chip having an immobilized protein other than transferrin receptor and deducting the change in the resonance unit when the sample specimen is allowed to flow onto this chip to exclude a so-called bulk effect by a solvent or the like. The Anti-TfR antibody (AbD Serotec catalogue number MCA1148) was diluted to 10 μg.mL⁻¹ in 10 mM sodium acetate pH 5.0 (GE Healthcare catalogue number BR-1003-50) and injected over flow cell 2 only. Whereas 50 μL of the transferrin receptor (TfR) (AbD Serotec catalogue number 9110-300) (diluted in HBS-EP (10 mM HEPES, 150 mM NaCl, 3 mM EDTA, 0.005% surfactant P-20, pH 7.4) to 10-20 μg.mL⁻¹) was injected over both flow cells. Excess ester groups on sensor chip surface were deactivated using ethanolamine hydrochloride (1 M pH 8.5).

HBS-EP was used as running buffer and dilution buffer for interaction analysis. Purified iron-loaded recombinant transferrin (S415A, T613A) or purified iron-loaded recombinant transferrin (S415C, T613A) was diluted to 10 μg.mL⁻¹ and 50 μL injected over both flow cells. Replicates were carried out to ensure reproducibility and the prepared Biacore sensor chip surface was regenerated between addition purified recombinant transferrin variants by 8-12 s injections of 10 mM sodium acetate pH 4.5 (GE Healthcare catalogue number BR-1003-50) between sample injections. Up to three injections were made, as required until baseline was restored.

The receptor binding capability of purified iron-loaded recombinant transferrin (S415C, T613A) appeared different to that of purified iron-loaded recombinant transferrin (S415A, T613A) by SPR analysis. Purified iron-loaded recombinant transferrin (S415A, T613A) gave a maximum response 59.3 (n=3), whereas purified iron-loaded recombinant transferrin (S415C, T613A) gave a maximum response 44.6 (n=3) indicating that functional differences in the transferrin receptor binding capability of recombinant transferrin (S415A, T613A) compared to recombinant transferrin (S415C, T613A). This indicated that the recombinant transferrin (S415A, T613A) mutant was preferred for the control of N-linked glycosylation.

### Example 7

### Mass Spectrometric Analysis of Recombinant Transferrin (S415A, T613A) Compared to Recombinant Transferrin (S32A. S415A, T613A) and Recombinant Transferrin (S32C, S415A, T613A)

ESI-TOF mass spectrometric analysis is a powerful method of studying post-translational changes and other modifications in proteins. It can provide mass accuracy of ±0.01% (down to a few Daltons in the case of transferrin) and is able to differentiate between species differing by as little as 20 Daltons.

Samples of recombinant transferrin (S32C, S415A, T613A), recombinant transferrin (S32A, S415A, T613A) were analysed by ESITOF mass spectrometry and compared to that of purified recombinant transferrin (S415A, T613A).

Samples of recombinant transferrin (S32C, S415A, T613A) and recombinant transferrin (S415A, T613A) were purified from high cell density fed-batch fermentation of Strain 1 [pDB3778] and Strain 1 [pDB3237] respectively, whereas, recombinant transferrin (S32A, S415A, T613A) was purified from Strain 1 [pDB3768] shake flask supernatant by concentrating 15 mL of the supernatant through a 10000 Da molecular weight cut-off spin column (Sartorius Vivaspin20-10000MWC0). The recombinant transferrin (S32A, S415A, T613A) sample was centrifuged according to the manufacturer's instructions, and then re-equilibrated using 15 mL 0.1% Trifluoracetic acid (TFA). The recombinant transferrin (S32A, S415A, T613A) sample was resuspended in 1.2 mL of 0.1% TFA and transferred to a microfuge tube prior to HPLC desalting. 0.5 mL of the recombinant transferrin (S32A, S415A, T613A) sample was desalted/concentrated using reversed phase-HPLC(RP.HPLC).

All samples were prepared for mass spectrometry as aqueous solutions of test proteins which were desalted/concentrated using RP.HPLC with recovered protein at concentrations of typically 20-100 nmol.mL⁻¹. The RP.HPLC desalting was carried on a Brownlee Aquapore BU-300(C4)7 mm, 100×2.1 mm column, the method utilised a binary gradient of 0.1% (v/v) Trifluoracetic acid (TFA) as solvent A and 70% (v/v) acetonitrile, 0.1% (v/v) TFA as solvent B with collection of eluting components detected by UV absorbance at 280 nm. Time-of-Flight mass spectrometry: Samples were introduced into a hybrid quadrupole time-of flight mass spectrometer (QqOaTOF, Applied Biosystems, QSTAR-XL®), equipped with an IonSpray™ source in positive ion mode, using flow injection analysis (FIA). The only instrument parameter that was actively tuned was the Decoupling Potential (DP) this was typically set to 250V. Typically 2 minutes of sample scans were averaged. For protein analysis the TOF analyser was calibrated against protonated molecular ions of equine myoglobin (Sigma) and resolution was typically 12,000. Instrument control and data acquisition and processing were performed using Analyst™ QS v1.1 software (Applied Biosystems).

Mass spectrometric analysis of transferrin (S415A, T613A) shows two peaks (FIG. 23). In this case one peak (marked UK in FIG. 23) is that corresponding to the unmodified transferrin (S415A, T613A) molecule with a nominal mass of 75097 (theoretical mass 75098 Da) (Spectrum 1 in FIG. 23). There is also a large peak (marked “B” in FIG. 23) with the expected 162 Dalton increment for a single hexose addition. This probably represents O-linked glycosylation. Mass spectrometric analysis of recombinant transferrins which have a mutation at serine-32 show only one peak. Mass spectrometric analysis of transferrin (S32C, S415A, T613A) shows only one main peak. The peak marked “C” in FIG. 23 is that corresponding to the unmodified transferrin (S32C, S415A, T613A) molecule with a nominal mass of 75112 (theoretical mass 75114 Da) (Spectrum 2 in FIG. 23). Furthermore mass spectrometric analysis of transferrin (S32A, S415A, T613A) shows only one main peak. The peak marked “D” in FIG. 23 is that corresponding to the unmodified transferrin (S32A, S415A, T613A) molecule with a nominal mass of 75082 (theoretical mass 75080 Da) (Spectrum 3 in FIG. 23). This result indicated that mutation of serine-32 prevented O-linked glycosylation at this position.

### Example 8

### Concanavalin A Analysis of Recombinant Transferrin (S415A, T613A) Compared to Recombinant Transferrin (S32A, S415A, T613A) and Recombinant Transferrin (S32C, S415A, T613A)

Concanavalin A (ConA) has been widely used in the study of glycoproteins due to its high affinity for oligosaccharide chains containing alpha-mannose residues. Purified samples of recombinant transferrin (S415A, T613A), recombinant transferrin (S32A, S415A, T613A) and recombinant transferrin (S32C, S415A, T613A) were subjected to ConA sepharose affinity chromatography; the concentration of the loaded sample and the eluate was determined by RP.HPLC allowing the % ConA binding material to be calculated (Table 4).

TABLE 4
Concanavalin A analysis of recombinant transferrin (S415A, T613A)
compared to recombinant transferring (S32A, S415A, T613A) and
ecombinant transferring (S32C, S415A, T613A).

| | | | | | | | Analysis | |
|---|---|---|---|---|---|---|---|---|
| | Load | Load | Load | Eluate | Eluate | Eluate | 1 Eluate | |
| | Volume | Conc. | Total | Volume | Conc. | Total | Recovery | Analysis 2 Eluate |
| Sample | (mL) | mg · mL⁻¹ | (mg) | (mL) | mg · mL⁻¹ | (mg) | % (w/w) | Recovery % (w/w) |
| Transferrin (S32C, S415A, T613A) | 10 | 10.45 | 104.50 | 6 | 0.044 | 0.26 | 0.25 | 0.26 |
| Transferrin (S32A, S415A, T613A) | 10 | 9.88 | 98.80 | 6 | 0.024 | 0.14 | 0.15 | — |
| Transferrin (S415A, T613A) Sample 1 | 1 | 10.27 | 10.27 | 6 | 0.148 | 0.89 | 8.65 | 6.19 |
| Transferrin (S415A, T613A) Sample 2 | 1 | 12.88 | 12.88 | 6 | 0.138 | 0.83 | 6.43 | — |
| Transferrin (S415A, T613A) Sample 3 | 1 | 15.15 | 15.15 | 6 | 0.135 | 0.81 | 5.35 | — |
| Transferrin (S415A, T613A) Sample 4 | 1 | 11.26 | 11.26 | 6 | 0.123 | 0.74 | 6.55 | 7.36 |
| Transferrin (S415A, T613A) Sample 5 | 1 | 13.87 | 13.87 | 6 | 0.097 | 0.58 | 4.20 | 5.78 |
| Transferrin (S415A, T613A) Sample 6 | 1 | 11.57 | 11.57 | 6 | 0.120 | 0.72 | 6.22 | 7.00 |

ConA columns were prepared by dispensing 4 mL 50% (v/v) slurry ConA sepharose beads:ConA equilibration buffer (100 mM NaOAc, 100 mM NaCl, 1 mM MgCl₂, 1 mM MnCl₂, 1 mM CaCl₂ pH 5.5) to 2 mL disposable columns. Transferrin samples (approximately 20 mg.mL⁻¹) were prepared at approximately 10 mg.mL⁻¹ by 1:1 dilution in ConA dilution buffer (200 mM NaOAc, 85 mM NaCl, 2 mM MgCl₂, 2 mM MnCl₂, 2 mM CaCl₂, pH 5.5). The concentrations of the diluted ‘load’ samples were confirmed by RP.HPLC. ConA columns were drained and equilibrated with 5 mL ConA equilibration buffer (100 mM NaOAc, 100 mM NaCl, 1 mM MgCl₂, 1 mM MnCl₂, 1 mM CaCl₂ pH 5.5).

1 ml of recombinant transferrin (S415A, T613A) was loaded onto a 2 mL ConA column, whereas 10 mL recombinant transferrin (S32A, S415A, T613A) and recombinant transferrin (S32C, S415A, T613A) were loaded onto a 2 mL ConA column. Columns were washed three times with ConA equilibration buffer, and eluted with 6 mL ConA elution buffer (100 mM NaOAc, 100 mM NaCl, 0.5 M Methyl-α-D-Mannopyranoside, pH 5.5). The concentrations of the eluted samples were determined by RP.HPLC (Table 4).

Approximately 6.6% recombinant transferrin (S415A, T613A) (n=10) binds to ConA, whereas only 0.25% (n=2) recombinant transferrin (S32C, S415A, T613A) and 0.15% (n=1) recombinant transferrin (S32A, S415A, T613A) was able to bind ConA demonstrating that mutation of serine-32 in transferrin causes a reduction in O-linked glycosylation, and that recombinant transferrin (S32A, S415A, T613A) is the preferred mutant for controlling glycosylation of the recombinant product.

1. A recombinant protein comprising the sequence of a transferrin mutant, wherein Ser415 is mutated to an amino acid which does not allow glycosylation of the transferrin mutant at Asn413.

2. A recombinant protein according to claim 1, wherein Ser415 is mutated to an amino acid that does not substantially reduce the biological function of the transferrin mutant.

3. A recombinant protein according to claim 1, wherein Ser415 is mutated to a conserved amino acid.

4. A recombinant protein according to claim 3, wherein Ser415 is mutated to alanine or glycine.

5. A recombinant protein comprising the sequence of a transferrin mutant, wherein Thr613 is mutated to an amino acid which does not allow glycosylation of the transferrin mutant at Asn611.

6. A recombinant protein according to claim 5, wherein Thr613 is mutated to an amino acid that does not substantially reduce biological function of the transferrin mutant.

7. A recombinant protein according to claim 5, wherein Thr613 is mutated to a conserved amino acid.

8. A recombinant protein according to claim 5, wherein Thr613 is mutated to glycine, valine, alanine or methionine.

9. A recombinant protein according to claim 8, wherein Thr613 is mutated to alanine.

10. A recombinant protein in accordance with claim 1, wherein Thr613 is mutated to an amino acid which does not allow glycosylation of the transferrin mutant at Asn611.

11. A recombinant protein according to claim 1, further comprising Asn611 mutated to an amino acid which does not allow glycosylation of the transferrin mutant at that location.

12. A recombinant protein according to claim 11, wherein Asn611 is mutated to an amino acid which does not substantially reduce biological function of the transferrin mutant.

13. A recombinant protein according to claim 11, wherein Asn611 is mutated to a conserved amino acid.

14. A recombinant protein according to claim 11, wherein Asn611 is mutated to aspartic acid or glutamine.

15. A recombinant protein according to claim 1, further comprising Val612 mutated to an amino acid which does not allow glycosylation of the transferrin mutant at Asn611.

16. A recombinant protein according to claim 15, wherein the amino acid does not substantially reduce the biological function of the transferrin mutant.

17. A recombinant protein according to claim 15, wherein Val612 is mutated to proline, tryptophan or cysteine.

18. A recombinant protein according to claim 5, wherein Asn413 is mutated to an amino acid which does not allow glycosylation of the transferrin mutant at that location.

19. A recombinant protein according to claim 18, wherein Asn413 is mutated to an amino acid which does not substantially reduce biological function of the transferrin mutant.

20. A recombinant protein according to claim 18, wherein Asn413 is mutated to a conserved amino acid, aspartic acid or glutamine.

21. A recombinant protein according to claim 5, further comprising Lys414 mutated to an amino acid which does not allow glycosylation of the transferrin mutant at Asn413.

22. A recombinant protein according to claim 21, wherein Lys414 is mutated to an amino acid which does not substantially reduce biological function of the transferrin mutant.

23. A recombinant protein according to claim 21, wherein Lys414 is mutated to proline, tryptophan or cysteine.

24. A recombinant protein comprising the sequence of a transferrin mutant, which transferrin mutant has the sequence defined by SEQ ID NO: 2.

25. A recombinant transferrin mutant consisting of the sequence defined by SEQ ID NO: 2.

26. A recombinant protein according to claim 1, wherein the transferrin further comprises at least one mutation that reduces the O-linked glycosylation.

27. A recombinant protein according to claim 26, wherein the at least one mutation that reduces O-linked glycosylation is a mutation corresponding to Ser32 in SEQ ID NO: 1, preferably S32A or S32C.

28. A polynucleotide comprising a sequence that encodes a protein comprising the sequence of a transferrin mutant in accordance with claim 1.

29. A polynucleotide according to claim 28 which comprises the sequence of SEQ ID NO: 3.

30. A polynucleotide according to claim 28 wherein the sequence that encodes a recombinant protein comprising the sequence of a transferrin mutant is operably linked to a polynucleotide sequence that encodes a secretion leader sequence.

31. A polynucleotide according to claim 30 wherein the sequence that encodes a recombinant protein comprising the sequence of a transferrin mutant is operably linked, at its 5′ end, to the 3′ end of a polynucleotide sequence that encodes a secretion leader sequence.

32. A plasmid comprising a polynucleotide according to claim 28.

33. A plasmid according to claim 32 which further comprises a polynucleotide sequence that encodes protein disulphide isomerise.

34. A plasmid according to claim 32 which is a S. cerevisiae 2 μm plasmid.

35. (canceled)

36. A method of producing a host cell capable of expressing a recombinant protein comprising the sequence of a transferrin mutant in accordance with claim 1 comprising:
(a) providing a polynucleotide as defined in any one of claims 28;
(b) providing a host cell;
(c) transforming the host cell with the polynucleotide; and
(d) selecting for a transformed host cell.

37. A method of producing a recombinant protein comprising the sequence of a transferrin mutant comprising:
(a) providing a host cell containing a polynucleotide comprising a sequence that encodes a protein comprising the sequence of a transferrin mutant in accordance with claim 1; and
(b) culturing the host cell under conditions that allow for the expression of the recombinant protein.

38. A method according to claim 37 further comprising the step of isolating the expressed recombinant protein.

39. (canceled)

40. (canceled)

41. (canceled)

42. A mammalian cell culture medium comprising a recombinant protein comprising the sequence of a transferrin mutant as claimed by claim 1 and one or more components selected from the group consisting of; glutamine, insulin, insulin-like growth factors, albumin, ethanolamine, fetuin, vitamins, lipoprotein, fatty acids, amino acids, sodium selenite, peptone and antioxidants.

43. A method of culturing mammalian cells, said method comprising incubating the cells in a cell culture medium comprising a recombinant protein comprising the sequence of a transferrin mutant as claimed by claim 1 and one or more components selected from the group consisting of; glutamine, insulin, insulin-like growth factors, albumin, ethanolamine, fetuin, vitamins, lipoprotein, fatty acids, amino acids, sodium selenite, peptone and antioxidants.

44. A composition comprising a recombinant protein according to claim 1.

45. A pharmaceutical composition comprising a recombinant protein comprising the sequence of a transferrin mutant as claimed by claim 1 and a pharmaceutically acceptable carrier.
